SEQUENCE LISTING (13 sequences)

SEQ ID NO: 1 - DNA, 11 bp, Unknown
  Unique binding site for restriction endocnuclease
ccnnnnnnngg

SEQ ID NO: 2 - DNA, 11 bp, unknown
  Unique binding site for restriction endocnuclease
ccnnnnnnngg

SEQ ID NO: 3 - DNA, 11 bp, Unknown
  Recognition sequence for restriction enzyme Ahd I
gacnnnnngtc

SEQ ID NO: 4 - DNA, 11 bp, unknown
  Recognition sequence for restriction enzyme Bgl I
gccnnnnnggc

SEQ ID NO: 5 - DNA, 11 bp, unknown
  Recognition sequence for restriction enzyme EcoN I
cctnnnnnagg

SEQ ID NO: 6 - DNA, 10 bp, UNKNOWN
  Recognition sequence for restriction enzyme Xmn I
gaannnnttc

SEQ ID NO: 7 - DNA, 130 bp, Artificial Sequence
  Internal primer used in amplification reaction
agcagaggcgcttctccgtgtccaccttgcgcaacttgggcctgggcaagaagtcgctggagcagtgggtgaccgaggaggccgcctgcctttgtgccgccttcgccaaccactccggtgggtgatgggc

SEQ ID NO: 8 - DNA, 18 bp, Artificial Sequence
  External forward primer
gagaccagggggagcata

SEQ ID NO: 9 - DNA, 17 bp, Artificial Sequence
  External reverse primer
ggcgatcacgttgctca

SEQ ID NO: 10 - DNA, 29 bp, Artificial Sequence
  Internal forward primer
tgggcctggatgctaagtcgctggcccag

SEQ ID NO: 11 - DNA, 17 bp, Artificial Sequence
  Internal reverse primer
ggcctcctcggtccccc

SEQ ID NO: 12 - DNA, 47 bp, Artificial Sequence
  Final amplification product
tgggcctggatgctaagtcgctggcccagtgggggaccgaggaggcc

SEQ ID NO: 13 - DNA, 31 bp, Artificial Sequence
  Digestion product
tgggcctggatgctaagtcgctggcccagtg

(19) United States
(12) Patent Application Publication
(10) Pub. No.: US 2004/0058349 A1
(43) Pub. Date: Mar. 25, 2004

(54) Methods for identifying nucleotides at defined positions in target nucleic acids

(76) Inventors: Jeffrey Van Ness, Claremont, CA (US); David J. Galas, Claremong, CA (US); Lori K. Garrison, Claremont, CA (US)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 10/398,004

(22) PCT Filed: Oct. 1, 2001
(86) PCT No.: PCT/US01/30742

(51) Int. Cl. C12Q 1/68

(52) U.S. Cl. 435/6

(57) ABSTRACT

The identity of a nucleotide of interest in a target nucleic acid molecule is determined by combining the target with two primers, where the first primer hybridizes to and extends from a location 3′ of the nucleotide of interest in the target, so as to incorporate the complement of the nucleotide of interest in a first extension product. The second primer then hybridizes to and extends based on the first extension product, at a location 3′ of the complement of the nucleotide of interest, so as to incorporate the nucleotide of interest in a second extension product. The first primer then hybridizes to and extends from a location 3′ of the nucleotide of interest in the second extension product, so as to form, in combination with the second extension product, a nucleic acid fragment. The first and second primers are designed to incorporate a portion of the recognition sequence of a restriction endonuclease that recognizes a partially variable interrupted base sequence. i.e. a sequence of the form A-B-C where A and C are a number and sequence of bases essential for RE recognition, and B is a number of bases essential for RE recognition. The first primer incorporates the sequence A, the second primer incorporates the sequence C, and they are designed, in view of the target, to product a nucleic acid fragment where sequences A and C are separated by the bases B, where the nucleotide of interest is within region B. Action of the RE on the nucleic acid fragment provides a small nucleic acid fragment that is amendable to characterization, to thereby reveal the identity of the nucleotide of interest.

### BACKGROUND OF THE INVENTION

1. Technical Field

This invention relates to the field of molecular biology, more particularly to methods and compositions involving nucleic acids, and still more particularly to methods and compositions for identifying a particular nucleotide in a target nucleic acid.

2. Description of the Related Art

The chromosomal mapping and nucleic acid sequencing of each of the 80,000 to 100,000 human genes, achieved through the Human Genome Project, provides an opportunity for a comprehensive approach to the identification of nucleotide loci responsible for genetic disease. Many of the 150-200 common genetic diseases and ˜600-800 of the rarer genetic diseases are associated with one or more defective genes. Of these, more than 200 human diseases are known to be caused by a defect in a single gene, often resulting in a change of a single amino acid residue. (Olsen, “Biotechnology: An Industry Comes of Age” (National Academic Press. 1986)).

Mutations occurring in somatic cells may induce disease if the mutations affect genes involved in cellular division control, resulting in, for example, tumor formation. In the germline, loss-of-function mutations in many genes can give rise to a detectable phenotype in humans. The number of cell generations in the germline, from one gamete to a gamete in an offspring, may be around 20-fold greater in the male germline than in the female. In the female, an egg is formed after a second meiotic division and lasts for 40 years. Therefore the incidence of different types of germline mutations and chromosomal aberrations depends on the parent of origin.

A majority of mutations, germline or somatic, are of little consequence to the organism since most of the genome appears to lack coding function (about 94%). Even within exon regions, there is some tolerance to mutations both due to the degeneracy of the genetic code and because the amino acid substitutions may have only a slight influence on a protein's function. (See, e.g., Strong et al., N. Engl. J. Med. 325:1597 (1991)). With the development of increasingly efficient methods to detect mutations in large DNA segments, the need to predict the functional consequences (e.g., the clinical phenotype) of a mutation become more important.

While point mutations predominate among mutations in the human genome, individual genes may exhibit peculiar patterns of mutations and, accordingly, pose different diagnostic problems. In approximately 60% of cases of Duchenne muscular dystrophy, the mutation involves a deletion of a large segment of the gigantic dystrophine gene. The elucidated mutation causing the fragile X syndrome is characterized by an increased copy number of a particular repeated sequence (CCG)ₙ. Hereditarily unstable DNA of this type may prove to be a more general phenomenon in human disease than is generally recognized.

Molecular genetic techniques have not been employed to a significant extent in the diagnosis of chromosomal aberrations in genetic and malignant disease; cytogenetics remains the preferred technique to investigate these important genetic mechanisms. In an individual with one mutated copy of a tumor suppressor gene, the remaining normal allele may be replaced by a second copy of the mutant allele in one cell per 10³-10⁴. Mechanisms causing this replacement include chromosomal nondisjunction, mitotic recombination, and gene conversion. In contrast, independent mutations destroying the function of the remaining gene copy, are estimated to occur in one cell out of 10⁶.

Sensitive mutation detection techniques offer extraordinary possibilities for mutation screening. For example, analyses may be performed even before the implantation of a fertilized egg. (Holding et al., Lancet 3:532 (1989)). Increasingly efficient genetic tests may also permit screening for oncogenic mutations in cells exfoliated from the respiratory tract or the bladder in connection with health checkups. (Sidransky et al. Science 252:706, 1991). Alternatively, when an unknown gene causes a genetic disease, methods to monitor DNA sequence variants are useful to study the inheritance of disease through genetic linkage analysis. Notwithstanding these unique applications for the detection of mutations in individual genes, the existing methodology for achieving such applications continues to pose technological and economic challenges. While several different approaches have been pursued, none are sufficiently efficient and cost effective for wide scale application.

Conventional methods for detecting mutations at defined nucleotide loci involve time-consuming linkage analyses in families using limited sets of genetic markers that are difficult to “readout.” Such methods include, e.g., DNA marker haplotyping (that identifies chromosomes with an affected gene) as well as methods for detecting major rearrangements such as large deletions, duplications, translocations and single base pair mutations. These methods include scanning, screening and fluorescence resonance energy transfer (FRET)-based techniques. (See, Cotton. “Mutation Detection” (Oxford University Press. 1997)).

Highly sensitive assays that detect low abundance mutations rely on PCR to amplify the target sequence. Non-selective PCR strategies, however, amplify both mutant and wild-type alleles with approximately equal efficiency. Accordingly, low abundance mutant alleles are represented in only a small fraction of the final product. Thus, if the mutant sequence comprises <25% of the amplified product, it is unlikely that DNA sequencing approaches will be able to detect its presence. Although it is possible to quantify low abundance mutations by first separating the PCR products by cloning and subsequent probing of the clones with allele-specific oligonucleotides (ASOs), this approach is both labor intensive (requiring multiple lengthy procedures) and costly. (Saiki et al., Nature 324:163-166 (1986); Sidransky et al. Science 256:102-105 (1992); and Brennan et al., N. Engl. J. Med. 332:429-435 (1995)).

In contrast to the above, allele-specific PCR methods can rapidly and preferentially amplify mutant alleles. For example, multiple mismatch primers have been used to detect H-ras mutations at a sensitivity of one mutant in 10⁵ wild-type alleles and sensitivity as high as one mutant in 10⁶ wild-type alleles have been reported. (Haliassos et al., Nucleic Acids Res. 17:8093-8099 (1989) and Chen et al., Anal. Biochem 244:191-194 (1997)). These successes are, however, limited to allele-specific primers discriminating through 3′ purine•purine mismatches. For the more common transition mutations, the discriminating mismatch on the 3′ primer end (i.e., G:T or C:A mismatch) will be removed in a small fraction of products by polymerase error during extension from the opposite primer on wild-type DNA. Thereafter, these error products are efficiently amplified and generate false positive signals.

It has been suggested that one means to eliminate the polymerase error problem is to deplete wild-type DNA early in the amplification cycles. Several reports have explored selective removal of wild-type DNA by restriction endonuclease digestion in order to enrich for low abundance mutant sequences. These restriction fragment length polymorphism (RFLP) methods detect approximately one mutant in 10⁶ wild-type or better. One approach has employed digestion of genomic DNA followed by PCR amplification of the uncut fragments (RFLP-PCR) to detect very low level mutations within restriction sites in the H-ras and p53 genes. (Sandy et al., Proc. Natl. Acad Sci. USA 89:890-894 (1992) and Pourzand et al., Mutat. Res. 288:113-121 (1993)). Similar results have been obtained by digestion following PCR and subsequent amplification of the un-cleaved DNA now enriched for mutant alleles (PCR-RFLP). (Kumar et al. Oncogene 3:647-651 (1988): Kumar et al. Oncogene Res. 4:235-241 (1989) and Jacobson et al. Oncogene 9:553-563 (1994)).

Although sensitive and rapid, RFLP detection methods are limited by the requirement that the location of the mutations must coincide with restriction endonuclease recognition sequences. To circumvent this limitation, primers that introduce a restriction site (part of the recognition sequence is in the template DNA) have been employed in “primer-mediated RFLP.” (Jacobson et al. PCR Methods Applicat. 1:299 (1992); Chen et al. Anal Biochem 195:51-56 (1991); Di Giuseppe et al. Am. J. Pathol. 144:889-895 (1994); Kahn et al. Oncogene 6:1079-1083 (1991); Levi et al. Res. 51 Cancer Res. 51:3497-3502 (1991) and Mitsudomi et al. Oncogene 6:1353-1362 (1991)). Subsequent investigators have demonstrated, however, that errors are produced at the very next base by polymerase extension from primers having 3′ natural base mismatches. (Hattori et al., Biochem Biophyis. Res. Commun 202:757-763 (1994); O'Dell et al. Genome Res. 6:558-568 (1996) and Hodanova et al. J. Inherit. Metab. Dis. 20:611-612 (1997)). Such templates fail to cleave during restriction digestion and amplify as false positives that are indistinguishable from true positive products extended from mutant templates.

Use of nucleotide analogs may reduce errors resulting from polymerase extension and improve base conversion fidelity. Nucleotide analogs that are designed to base pair with more than one of the four natural bases are termed “convertides.” Base incorporation opposite different convertides has been tested. (Hoops et al., Nucleic Acids Res. 25:4866-4871 (1997)). For each analog, PCR products were generated using Taq DNA polymerase and primers containing an internal nucleotide analog. The products generated showed a characteristic distribution of the four bases incorporated opposite the analogs.

Due, in part, to the shortcomings in the existing methodology for detecting genetic mutations, there exists an unmet need for rapid and sensitive methods for detecting mutations at defined nucleotide loci within target nucleic acids. The present invention fulfills this and other related needs by providing methods for the detection of mutations at defined nucleotide loci in target nucleic acids that, inter alia, display increased speed, convenience and specificity. As disclosed in detail herein below, methods according to the present invention are based on the incorporation of unique restriction endonuclease restriction sites flanking and/or encompassing the mutant nucleotide loci. These methods exploit the high degree of specificity afforded by restriction endonucleases and employ readily available detection techniques.

### SUMMARY OF THE INVENTION

The present invention provides various compounds and compositions useful for, and method of, identifying single nucleotide polymorphisms at defined positions in target nucleic acids.

In one aspect, the present invention provides a method for identifying a nucleotide at a defined position in a single-stranded target nucleic acid, comprising the following steps:

(a) forming a mixture of a first oligonucleotide primer (ODNP), a second ODNP, and the target nucleic acid, wherein

the first ODNP comprises a nucleotide sequence that is complementary to a nucleotide sequence of the target nucleic acid at a location 3′ to the defined position.

the second ODNP comprises a nucleotide sequence that is complementary to a nucleotide sequence of the complement of the target nucleic acid at a location 3′ to the complementary nucleotide of the nucleotide at the defined position, and

the first and second ODNPs further comprise a first constant recognition sequence (CRS) of a first strand and a second CRS of a second strand of an interrupted restriction endonuclease recognition sequence (IRERS), respectively, but not a complete IRERS, the complete IRERS being a double-stranded nucleic acid having the first and the second strands and comprising the first and the second constant recognition sequences (CRS) linked by a variable recognition sequence (VRS);

(b) extending the first and second ODNPs to form a fragment having the complete IRERS wherein the nucleotide to be identified is within the VRS;

(c) cleaving the fragment with a restriction endonuclease that recognizes the complete IRERS; and

(d) characterizing a product of step (c) to thereby determine the identity of the nucleotide at the defined position.

In some embodiments, the defined position may be polymorphic or associated with a disease, including a human genetic disease (e g., bladder carcinoma, colorectal tumors, sickle-cell anemia, thalassemias, al-antitrypsin deficiency, Lesch-Nyhan syndrome, cystic fibrosis/mucoviscidosis, Duchenne/Becker muscular dystrophy, Alzheimer's disease, X-chromosome-dependent mental deficiency, and Huntington's chorea, phenylketonuria, galactosemia, Wilson's disease, hemochromatosis, severe combined immunodeficiency, alpha-1-antitrypsin deficiency, albinism, alkaptonuria, lysosomal storage diseases. Ehlers-Danlos syndrome, hemophilia, glucose-6-phosphate dehydrogenase disorder, agammaglobulimenia, diabetes insipidus, Wiskott-Aldrich syndrome, Fabn's disease, fragile X-syndrome, familial hypercholesterolemia, polycystic kidney disease, hereditary spherocytosis. Marfan's syndrome, von Willebrand's disease, neurofibromatosis, tuberous sclerosis, hereditary hemorrhagic telangiectasia, familial colonic polyposis, Ehlers-Danlos syndrome, myotonic dystrophy, osteogenesis imperfecta, acute intermittent porphyria, and von Hippel-Lindau disease). In other embodiments, a mutation at the defined position is associated with drug resistance of a pathogenic microorganism.

In certain embodiments, the single-stranded target nucleic acid may be one strand of a denatured double-stranded nucleic acid, such as genomic nucleic acid and cDNA. In other embodiments, the single-stranded target nucleic acid may be derived from the genome of a pathogenic virus or from the genome or episome of a pathogenic bacterium. In yet other embodiment, the target nucleic acid is synthetic nucleic acid.

In some embodiments, either the nucleotide sequence of the first ODNP complementary to the target nucleic acid, or the nucleotide sequence of the second ODNP complementary to the complement of the target nucleic acid, or both are at least 6, 8, 10, 12, 14 or 16 nucleotides in length.

In certain embodiments, either the first ODNP, or the second ODNP, or both ODNPs are 8-100 nucleotides in lengths more preferably 15-85 nucleotides in length. The first ODNP may further comprise one or more nucleotides complementary to the target nucleic acid at the 3′ terminus of the first CRS. Similarly, the second ODNP may further comprise one or more nucleotides complementary to the target nucleic acid at the 3′ terminus of the second CRS.

In certain embodiments, step (b) of the present method comprises performing a polymerase chain reaction. In some embodiments, step (d) may be performed at least partially by the use of mass spectrometry, liquid chromatography, fluorescence polarization, electron ionization, gel electrophoresis, or capillary electrophoresis. In addition, all of steps (a) through (d) may be performed in a single vessel.

In a preferred embodiment, the IRERS is recognizable by Bsl I, Mwo I, and Xcm I.

Another aspect of the present invention provides an oligonucleotide primer, comprising

(a) a first CRS of a first strand of an IRERS, but not the first strand of a complete IRERS, the complete IRERS being a double-stranded oligonucleotide having the first strand and a second strand and comprising the first CRS and a second CRS linked by a VRS, the VRS having a number n of variable nucleotides; and

(b) at a location 5′ to the 5′ terminus of the first CRS, an oligonucleotide sequence complementary to a nucleotide sequence of a single-stranded target nucleic acid at a location 3′ to a defined position, wherein when the oligonucleotide sequence anneals to the target nucleic acid, the distance between the nucleotide in the target corresponding to the 3′ terminal nucleotide of the primer and the defined position is within the range 0 to n-1.

In certain embodiments, oligonucleotide sequence (b) is at least 6, 8, 10, 12, 14, or 16 nucleotides in length. In some embodiments, the primer is 8-200 nucleotides in length. In other preferred embodiments, the primers are 15-85 or 18-32 nucleotide in length. The primer may further comprise one or more nucleotides complementary to the target nucleic acid at the 3′ terminus of the first CRS. In a preferred embodiment, the IRERS is recognizable by Bsl I.

Preferably, the defined position in the target nucleic acid is polymorphic. In some embodiments, a mutation at the defined position in the target nucleic acid is associated with a disease. The target nucleic acid may one strand of a denatured double-stranded nucleic acid, including genomic nucleic acid and cDNA.

Another aspect of the present invention provides an oligonucleotide primer pair for producing a portion of a single-stranded target nucleic acid containing a nucleotide to be identified at a defined position. Such a primer pair comprise first and second ODNPs wherein (1) the first ODNP comprises a nucleotide sequence complementary to a nucleotide sequence of the target nucleic acid at a location 3′ to the defined position; (2) the second ODNP comprises a nucleotide sequence complementary to a nucleotide sequence of the complement of the target nucleic acid at a location 3′ to the complementary nucleotide of the nucleotide to be identified; (3) the first and second ODNPs further comprise a first constant recognition sequence (CRS) of a first strand and a second CRS of a second strand of an interrupted restriction endonuclease recognition sequence (IRERS), respectively, but not a complete IRERS, the complete IRERS being a double-stranded nucleic acid having the first and the second strands and comprising the first and the second constant recognition sequences (CRS) linked by a variable recognition sequence (VRS); and (4) a fragment resulting from an amplification of the first and second ODNPs comprises a complete IRERS, wherein the nucleotide to be identified is within the VRE.

In some embodiments, either the nucleotide sequence complementary to the target nucleic acid of the first ODNP, or the nucleotide sequence complementary to the complement of the target nucleic acid of the second ODNP, or both, are at least 6, 8, 10, 12, 14, or 16 nucleotides in length. Preferably, the IRERS is recognizable by Bsl I.

In certain embodiments, either the first ODNP, or the second ODNP, or both ODNPs are 8-100 nucleotides in length, preferably 15-85 nucleotides in length. Preferably, the first ODNP may further comprise one or more nucleotides complementary to the target nucleic acid at the 3′ terminus of the first CRS. Likewise, the second ODNP may further comprise one or more nucleotides complementary to the target nucleic acid at the 3′ terminus of the second CRS.

The defined position in the target nucleic acid may be polymorphic or associated with a disease. The target nucleic acid may be one strand of a denatured double-stranded nucleic acid, such as genomic nucleic acid and cDNA.

The present invention provides a composition comprising the primer and the target nucleic acid as described above. It further provides a kit comprising the above primer pair. The kit may further comprise a restriction endonuclease that recognizes the IRERS a portion of which constitutes partial sequences of the primer pair. The kit may also further comprise instruction of use thereof.

In another aspect, the present invention provides a set of two ODNP pairs, comprising first and second ODNP pairs each comprising first and second ODNPs wherein:

(a) the first ODNP in the first ODNP pair comprises

an oligonucleotide sequence complementary to a nucleotide sequence of a single-stranded target nucleic acid at a location 3′ to a defined position in the target nucleic acid, and

a first CRS of a first strand of an IRERS, but not the first strand of a complete IRERS, the complete IRERS being a double-stranded nucleic acid having first and second strands and comprising the first CRS and a second CRS linked by a VRS;

(b) the second ODNP in the first ODNP pair comprises

an oligonucleotide sequence complementary to a nucleotide sequence of the target nucleic acid at a location 5′ to the defined position, and

a second CRS of the first strand of the IRERS, but not the first strand of the complete IRERS;

(c) the first ODNP in the second ODNP pair comprises

an oligonucleotide sequence complementary to a nucleotide sequence of the complement of the target nucleic acid at a location 5′ to the position in the complement corresponding to the defined position in the target nucleic acid, and

a first CRS of the second strand of the IRERS, but not the second strand of the complete IRERS; and

(d) the second ODNP in the second ONDP pair comprises

an oligonucleotide sequence complementary to a nucleotide sequence of the complement of the target nucleic acid at a location 3′ to the position in the complement corresponding to the defined position in the target nucleic acid, and

a second CRS of the second strand of the IRERS, but not the second strand of the complete IRERS; and

(e) a fragment resulting from an extension and ligation of the first and second ODNPs in each ODNP pair comprises the complete IRERS, wherein the nucleotide to be identified is within the VRS.

In yet another aspect, the present invention provides a method comprising the following steps:

(a) providing a double-stranded nucleic acid molecule comprising an interrupted restriction endonuclease recognition sequence (IRERS), wherein the IRERS comprises a first constant recognition sequence (CRS) and a second CRS linked by a variable recognition sequence (VRS), the VRS having a nucleotide of interest;

(b) cleaving the nucleic acid molecule with a restriction endonuclease that recognizes the IRERS; and

(c) characterizing at least one of the products of step (b) to determine the identity of the nucleotide of interest.

In certain embodiments, at least one of the products of step (b) is characterized by a technique selected from liquid chromatograph, mass spectrometry) electron ionization, gel electrophoresis, and capillary electrophoresis. In some embodiment, the restriction endonuclease is Bsl I.

In some embodiments, step (a) comprises: (i) forming a mixture of the primer pair set and the target nucleic acid as described above; (ii) extending the first and second ODNPs of the first and second ODNP pairs; (iii) ligating the extended products of step (b); and (iv) amplifying the fragments of step (c). In other embodiments, step (a) comprises: (i) forming a mixture of the primer pair described above and the target nucleic acid; and (ii) extending the first and the second ODNPs. In yet other embodiments, step (a) comprises: (i) forming a mixture of a first ODNP, a second ODNP and a single-stranded target, wherein (1) the first ODNP comprises an oligonucleotide sequence complementary, to a nucleotide sequence of the target nucleic acid at a location 3′ to a defined position in the a target nucleic acid and a first CRS of a first strand of an IRERS, but not the first strand of a complete IRERS, the complete IRERS being a double-stranded nucleic acid having first and second strands and comprising the first CRS and a second CRS linked by a VRS, and (2) the second ODNP comprises an oligonucleotide sequence complementary to a nucleotide sequence of the target nucleic acid at a location 5′ to the defined position and a second CRS of the first strand of the IRERS, but not the first strand of the complete IRERS: (ii) extending the first and second ODNPs; (iii) ligating the extended products of step (ii): and (iv) annealing the ligation product of step (iii) with an oligonucleotide wherein the oligonucleotide has a universe nucleotide at the position corresponding to the defined position in the target nucleic acid and the resulting double-stranded nucleic acid molecule comprising an IRERS.

In another aspect, the present invention provides a method comprising,

(a) combining a first ODNP, a second ODNP, and a target nucleic acid under primer extension conditions, wherein (1) the first ODNP comprises an oligonucleotide sequence complementary to a nucleotide sequence of the target nucleic acid at a location 3′ to a defined position in the a target nucleic acid and a first CRS of a first strand of an IRERS, but not the first strand of a complete IRERS, the complete IRERS being a double-stranded nucleic acid having first and second strands and comprising the first CRS and a second CRS linked by a VRS, and (2) the second ODNP comprises an oligonucleotide sequence complementary to a nucleotide sequence of the target nucleic acid at a location 5′ to the defined position and a second CRS of the first strand of the IRERS, but not the first strand of the complete IRERS;

(b) performing at least three rounds of primer extension to provide a primer extension product;

(c) cleaving the primer extension product with a restriction endonuclease that recognizes an interrupted restriction endonuclease recognition sequence (IRERS); and

(d) characterizing at least one of the products of step (c) by a technique selected from liquid chromatography, mass spectrometry, electron ionization, gel electrophoresis, and capillary eletrophoresis.

In certain embodiments, step (b) comprises performing a polymerase chain reaction. In some embodiments, the target nucleic acid is genomic DNA or cDNA. Preferably, all of steps (a) through (c) are performed in a single vessel. In a preferred embodiment, the restriction endonuclease is Bsl I.

### BRIEF DESCRIPTION OF THE SEVERAL VIEWS OF THE DRAWINGS

FIG. 1 is a diagram of major steps in a method of the present invention for identifying a nucleotide at a defined position in a target nucleic acid using an ONDP pair and an exemplary restriction endonuclease recognition sequence for Bsl I.

FIG. 2 is a schematic diagram of the major components of the ODNPs and a resulting amplicon of the present invention.

FIG. 3 is a schematic diagram of the major components of an interrupted restriction endonuclease recognition sequence. A₁A₂ . . . Aₘ is a specific nucleotide sequence consisting of m nuleotides, whereas A′₁A′₂ . . . A′ₘ is the complement sequence of A₁A₂ . . . Aₘ. The double-stranded fragment comprised of A₁A₂ . . . Aₘ and A′₁A′₂ . . . A′ₘ forms the first CRS (also referred to as “Region A”). N₁N₂ . . . Nₙ is a variable nucleotide sequence consisting of n nucleotides where any one of the nucleotide can contain any of the four bases (a, c, t, or g). N′₁N′₂ . . . N′ₙ is the complement of N₁N₂ . . . Nₙ and forms a VRS (also referred to “Region B” where the number n is equal to the number B) in combination of N₁N₂ . . . N_{n.} C₁C₂ . . . Cᵢ is a specific nucleotide sequence consisting of i nucleotides, whereas C′₁C′₂ . . . C′ᵢ is the complement of C₁C₂ . . . Cᵢ. The double-stranded fragment comprised of C₁C₂ . . . Cᵢ and C′₁C′₂ . . . C′ᵢ forms the second CRS (also referred to as “Region C”).

FIG. 4 is a schematic diagram of a set of two ODNP pairs.

FIG. 5 is a schematic diagram of major steps in the present method for identifying a nucleotide at a defined position in a target nucleic acid using a set of two ODNP pairs and the exemplary restriction endonuclease recognition sequence for Bsl I.

FIG. 6 is a schematic diagram of major steps of one embodiment of the present method for providing a double-stranded nucleic acid molecule containing an IRERS.

FIG. 7 shows the UV chromatograms. The top panel shows the genotyping fragment with an M/Z value of 1246 (8 charges) representing a wild type allele of cytochrome 2D6 gene. The second panel is the positive control of 1232 for (8 charges) to calibrate the M/Z measurements. The third panel is the UV trace and the bottom panel shows the total ion current.

### DETAILED DESCRIPTION OF THE INVENTION

The present invention provides methods, compositions, and kits for determining sequence information at a defined genetic locus in a target nucleic acid. As described in more detail below, the invention provides for the design, preparation and use of oligonucleotide primers (ODNPs) that can be extended in a manner that incorporates information about the nucleotide of interest into the extension product. The resulting product, e.g., amplicon, can then be analyzed by various methods, also described in more detail below, to determine the identity of the nucleotide of interest. This information is advantageously utilized in a variety of applications, as described herein, such as genetic analysis for hereditary diseases, tumor diagnosis, disease predisposition, forensics or paternity, crop cultivation and animal breeding, expression profiling of cell function and/or disease marker genes, and identification and/or characterization of infectious organisms that cause infectious diseases in plants or animals and/or that are related to food safety.

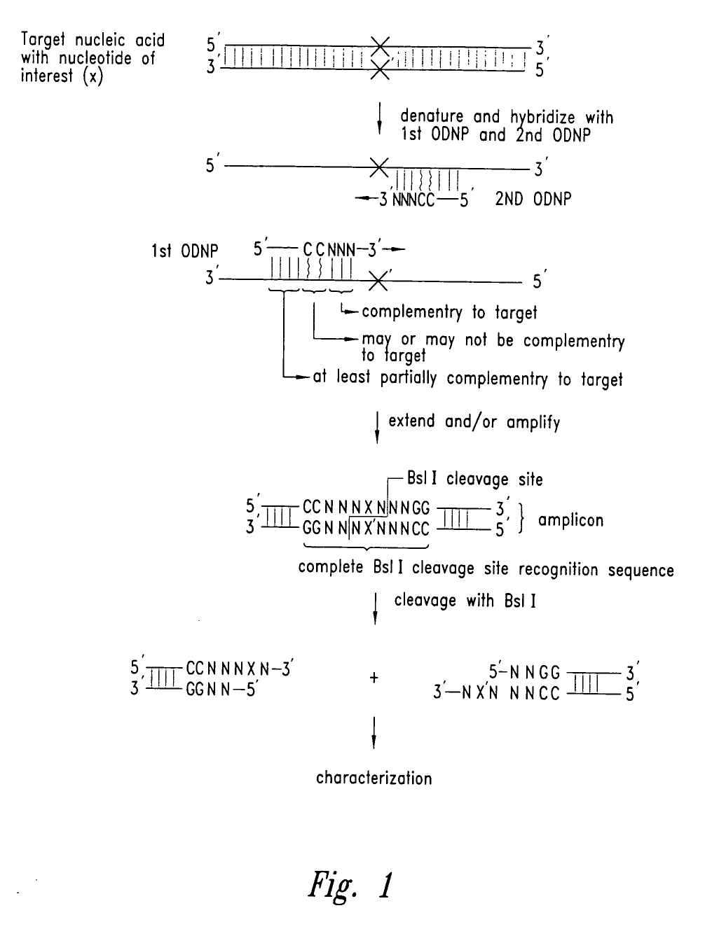

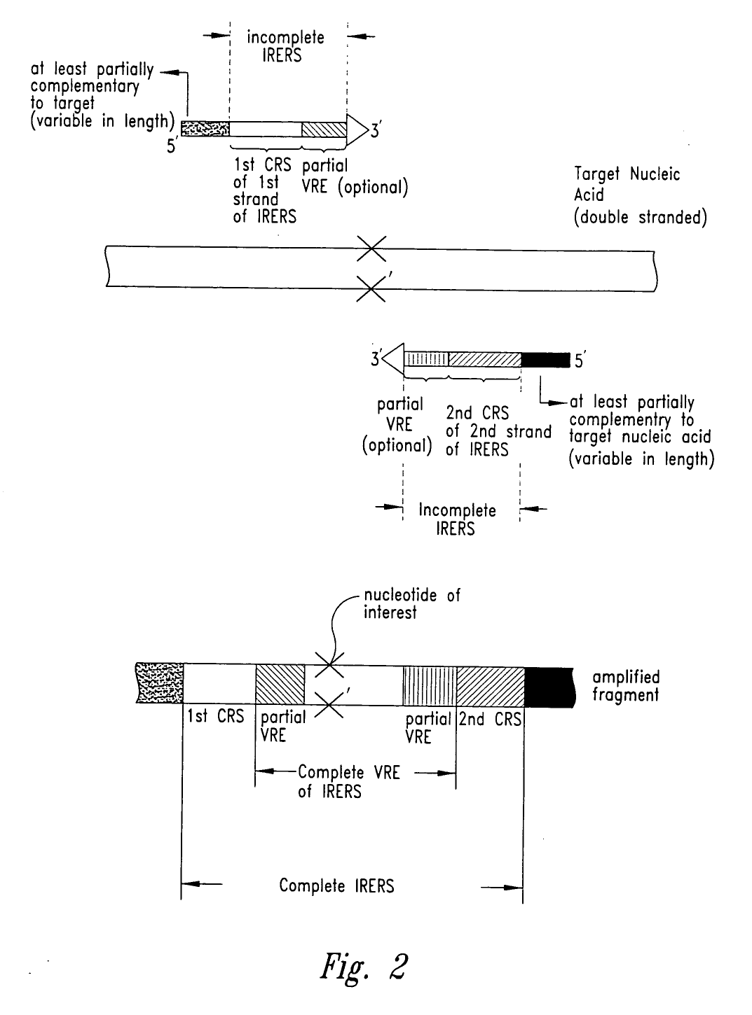

The ODNPs of the present invention each contain part of an interrupted restriction endonuclease recognition sequence (IRERS), defined in detail below. The interrupted segment of the restriction endonuclease recognition site (also referred to as “variable recognition sequence (VRS)”) may be one or more nucleotides in length and the sequence is variable (each position can contain any of the four bases (a, c, t, or g)). When extended and incorporated into an amplified fragment, the two primers together, in combination with the segment of target nucleic acid between them (i.e. VRS) form a single and complete IRERS. The primers are designed such that the nucleotide of interest in a target nucleic acid is located in the amplicon within the variable segment of the restriction endonuclease recognition site. The amplicon can then be digested to (generate small fragments of nucleic acid that can be analyzed to determine the nucleotide of interest with great accuracy and sensitivity. The oligonucleotide primers of the present invention are shown schematically in FIG. 2. In FIG. 1, a diagram of the present invention is shown using the exemplary restriction endonuclease recognition sequence for Bsl I. One skilled in the art will appreciate that any interrupted restriction endonuclease recognition sequence may be used (see Table 2).

In various aspects, the present invention provides assays for determining the identity of a base at a predetermined location in a target nucleic acid molecule. In additional aspects, provided herein are compounds and compositions that are useful in performing such assays. In other aspects, the present invention provides compounds and compositions that, upon suitable characterization, identify the base at a predetermined location in a target nucleic acid. Still further aspects of the present invention are described hereinbelow.

A. Conventions

Prior to providing a more detailed description of the present invention, it may be helpful to an understanding thereof to define a convention as used herein, as follows. The terms “3′” and “5′” are used herein to describe location of a particular site within a single strand of nucleic acid. When a location in nucleic acid is “3′ to” or “3′ of” a nucleotide of interest, this means that it is between the nucleotide of interest and the 3′ hydroxyl of that strand of nucleic acid. Likewise, when a location in a nucleic acid is “5′ to” or “5′ of” a nucleotide of interest, this means that it is between the nucleotide of interest and the 5′ phosphate of that strand of nucleic acid.

Also, as used herein, the word “a” refers to one or more of the indicated items. For instance, “a” polymerase refers to one or more polymerases.

B. Methodology of the Present Invention

1. Overview of the Methodology of the Present Invention

According to the present invention, the identity of a nucleotide of interest in a target nucleic acid molecule is determined by combining the target with two primers, where the first primer hybridizes to and extends from a location 3′ of the nucleotide of interest in the target, so as to incorporate the complement of the nucleotide of interest in a first extension product. The second primer then hybridizes to and extends based on the first extension product, at a location 3′ of the complement of the nucleotide of interest, so as to incorporate the nucleotide of interest in a second extension product. The first primer then hybridizes to and extends from a location 3′ of the nucleotide of interest in the second extension product, so as to form, in combination with the second extension product, a nucleic acid fragment. The first and second primers are designed to incorporate a portion of the recognition sequence of a restriction endonuclease that recognizes a partially variable interrupted base sequence, i.e., a sequence of the form A-B-C where A and C are a number and sequence of bases essential for RE recognition, and B is a number of bases essential for RE recognition. The first primer incorporates the sequence A, the second primer incorporates the sequence C, and they are designed, in view of the target, to product a nucleic acid fragment where sequences A and C are separated by the bases B, where the nucleotide of interest is within region B. Sequences in regions A, B and C are also referred to as “the first constant recognition sequence (CRS),” “variable recognition sequence,” and “the second CRS,” respectively. Action of the RE on the nucleic acid fragment provides a small nucleic acid fragment that is amendable to characterization, to thereby reveal the identity of the nucleotide of interest. The use of short nucleic acid (e.g., DNA) fragments is advantageous for numerous readout systems because amplicons produced during, e.g., a PCR amplification reaction, need not be tagged or labeled to facilitate detection.

Alternatively, a nucleotide at a defined position in a target nucleic acid is identified by combining the target with a set of two primer pairs. The first and second primers of the first primer pair hybridize to the target at a location 3′ and 5′ to the defined position, respectively. The first and second primers of the second primer pair hybridize to the complement of the target at a location 5′ and 3′ to the defined position, respectively. Each ODNP of the primer pair set is designed to incorporate a portion of an IRERS (i.e. CRS) so that the extension and/or amplification product of the primer pair set with the target as a template in the presence of a DNA polymerase and a DNA ligase contains the complete IRERS. The extension and/or amplification product is then digested with a RE that recognizes the IRERS and the resulting small fragment is characterized. The nucleotide at the defined position is thereby identified.

2. Target Nucleic Acid Molecules

Methods, kits and compositions of the present invention typically involve or include a target nucleic acid molecule. The target nucleic acid of the present invention is any nucleic acid molecule about which base information is desired, and which can serve as a template for a primer extension reaction. i.e., can base pair with a primer.

The term “nucleic acid” refers generally to any molecule, preferably a polymeric molecule, incorporting units of ribonucleic acid or an analog thereof. The template nucleic acid can be either single-stranded or double-stranded. A single-stranded template nucleic acid may be one strand nucleic acid of a denatured double-stranded DNA. Alternatively, it may be a single-stranded nucleic acid not derived from any double-stranded DNA. In one aspect, the template nucleic acid is DNA. In another aspect, the template is RNA. Suitable nucleic acid molecules are DNA, including genomic DNA, ribosomal DNA and cDNA. Other suitable nucleic acid molecules are RNA, including mRNA, rRNA and tRNA. The nucleic acid molecule may be naturally occurring, as in genomic DNA, or it may be synthetic. i.e., prepared based up human action, or may be a combination of the two.

A naturally occurring nucleic acid is obtained from a biological sample. Preferred biological samples include one or more mammalian tissues. (for example blood, plasma/serum, hair, skin, lymph node, spleen, liver, etc) and/or cells or cell lines. The biological samples may comprise one or more human tissues and/or cells. Mammalian and/or human tissues and/or cells may further comprise one or more tumor tissues and/or cells.

Methodology for isolating populations of nucleic acids from biological samples are well known and readily available to those skilled in the art of the present invention. Exemplary techniques are described, for example, in the following laboratory research manuals: Sambrook et al. “Molecular Cloning” (Cold Spring Harbor Press. 3rd Edition, 2001) and Ausubel et al. “Short Protocols in Molecular Biology” (1999) (incorporated herein by reference in their entirety). Nucleic acid isolation kits are also commercially available from numerous companies which simplify and accelerate the isolation process.

A synthetic nucleic acid is produced by human intervention. At this time, many companies are in the business of making and selling synthetic nucleic acids that may be useful as the template nucleic acid molecule in the present invention. See. e.g. Applied Bio Products Bionexus (www.bionexus.net); Commonwealth Biotechnologies, Inc. (Richmond, Va.; www.cbi-biotech.com): Gemini Biotech (Alachua. Fla.; www.geminibio.com); INTERACTIVA Biotechnologie GmbH (Ulm, Germany; www.interactiva.de); Microsynth (Balgachi. Switzerland; www.microsynth.ch); Midland Certified Reagent Company (Midland, Tex.; www.mcrc.com); Oligos Etc. (Wilsonville, Oreg.; www.oligosetc.com); Operon Technologies, Inc. (Alameda, Calif.; www.operon.com); Scandanavian Gene Synthesis AB (Koöping, Sweden; www.sgs.dna); Sigma-Genosys (The Woodlands, Tex.; www.genosys.com); Synthetic Genetics (San Digeo, Calif.; www.syntheticgenetics.com, which was recently purchased by Epoch Biosciences. Inc. (Bothell. Wash.; www.epochbio.com); and many others.

The synthetic nucleic acid template may be prepared using an amplification reaction. The amplification reaction may be, for example, the polymerase chain reaction.

The synthetic nucleic acid template may be prepared using recombinant DNA means through production in one or more prokaryotic or eukaryotic organism such as. e.g., E. coli. yeast. Drosophila or mammalian tissue culture cell line.

The nucleic acid molecule may, and typically will, contain one or more of the ‘natural’ nucleotides. i.e. adenine (A), guanine (G), cytosine (C), thymine (T) and, in the case of an RNA, uracil (U). In addition, and particularly when the nucleci acid is a synthetic molecule, the target nucleic acid may include “unnatural” nucleotides. Unnatural nucleotides are chemical moieties that can be substituted for one or more natural nucleotides in a nucleotide chain without causing the nucleic acid to lose its ability to serve as a template for a primer extension reaction. The substitution may include either sugar and/or phosphate substitutions, in addition to base substitutions.

Such moieties are very well known in the art, and are known by a large number of names including for example, abasic nucleotides, which do not contain a commonly recognized nucleotide base, such as adenine, guanine, cytosine, uracil or thymine (see, e.g., Takeshita et al. “Oliaonucleotides containing synthetic abasic sites” The Journal of Biological Chemistry, vol. 262, pp. 10171-10179 1987; Iyer et al. “Abasic oligodeoxyribonucleoside phosphorothioates: synthesis and evaluation as anti-HIV-1 agents “Nucleic acids Research, vol. 18, pp. 2855-2859 1990: and U.S. Pat. No. 6,117,657): base or nucleotide analogs (see, e.g. Ma et al. “Design and Synthesis of RNA Miniduplexes via a Synthetic Linker Approach. 2. Generation of Covalently Closed. Double-Stranded Cyclic HIV-1 TAR RNA Analogs with High Tat-Binding Affinity,” Nuicleic Acids Research 21:2585 (1993). Some bases are known as universal mismatch base analogs, such as the abasic 3-nitropyrrole), convertides (see, e.g. Hoops et al., Nucleic Acids Res. 25:4866-4871 (1997)); modified nucleotides (see, e.g., Millican et al. “Synthesis and biophysical studies of short oligodeoxynucleotides with novel modifications: A possible approach to the problem of mixed base oligodeoxynucleotide synthesis,” Nucleic Acids Research 12:7435-7453 (1984); nucleotide mimetics; nucleic acid related compounds; spacers (see. e.g., Nielsen et al. Science. 254:1497-1500 (1991); and specificity spacers (see, e.g., PCT International Publication No. WO 98/13527).

Additional examples of non-natural nucleotides are set forth in: Jaschke et al. Tetrahedron Lett. 34:301 (1993); Seela and Kaiser, Nucleic Acids Research 15:3113 (1990) and Nucleic Acids Research 18:6353 (1990); Usman et al., PCT International Patent Application No. PCT/US 93/00833; Eckstein, PCT International Patent Application No. PCT/EP91/01811; Sproat et al., U.S. Pat. No. 5,334,711, and Buhr and Matteucci, PCT International Publication No. WO 91/06556; Augustyns, K. A, et al. Nucleic Acids Res., 1991, 19, 2587-2593); and U.S. Pat. Nos. 5,959,099 and 5,840,876.

When the template nucleic acid molecular, and/or the primer used in the present method, contains a non-natural nucleotide, then a base-pair mismatch will occur between the template and the primer. The term “base-pair mismatch” refers to all single and multiple nucleotide substitutions that perturb the hydrogen bonding between conventional base pairs. e.g., G:C. A:T, or A:U, by substitution of a nucleotide with a moiety that does not hybridize according to the standard Watson-Crick model to a corresponding nucleotide on the opposite strand of the oligonucleotide duplex. Such base-pair mismatches include, e.g., G:G, G:T, G:A, G:U, C:C, C:A, C:T, C:U, T:T, T:U, U:U and A:A. Also included within the definition of base-pair mismatches are single or multiple nucleotide deletions or insertions that perturb the normal hydrogen bonding of a perfectly base-paired duplex. In addition, base-pair mismatches arise when one or both of the nucleotides in a base pair has undergone a covalent modification (e.g., methylation of a base) that disrupts the normal hydrogen bonding between the bases. Base-pair mismatches also include non-covalent modifications such as, for example, those resulting from incorporation of intercalating agents such as ethidium bromide and the like that perturb hydrogen bonding by altering the helicity and/or base stacking of an oligonucleotide duplex.

The template, in addition to containing nucleic acids or analogs thereof, also contains one or more natural bases of unknown identity. The present invention provides compositions and methods whereby the identity of the unknown nucleotide(s) becomes known. The base(s) of unknown identity is present at the “nucleotide loci” (or the “defined position”), refers to a specific nucleotide or region encompassing one or more nucleotides having a precise location on a target nucleic acid.

The base(s) to be identified in the target nucleic acid may be a mutation. The term “mutation” refers to an alteration in a wild-type nucleic acid sequence. Mutations may be in regions encoding proteins (exons) or may be in non-coding regions (introns or 5′ and 3′ flanking regions) of a target nucleic acid. Exemplary mutations in non-coding regions include regulatory mutations that alter the amount of gene product, localization of protein and/or timing of expression. The term point mutations” refers to mutations in which a wild-type base (i.e., A, C, G, or T) is replaced with one of the other bases at a defined nucleotide locus within a nucleic acid sample. They can be caused by a base substitution or a base deletion. A “frameshift mutation” is caused by a small deletion or insertion that, in turn, causes the reading frame to be shifted and, thus, a novel peptide to be formed. A “regulatory mutation” is a mutation in a region(s) of the gene not coding for protein, e.g., intron, 5′- or 3′-flanking, but affecting correct expression (e.g., amount of product, localization of protein, timing of expression). A “nonsense mutation” is a single nucleotide change resulting in a triplet codon (where mutation occurs) being read as a “STOP” codon causing premature termination of peptide elongation. i.e., a truncated peptide. A “missense mutation” is a mutation that results in one amino acid being exchanged for a different amino acid. Such a mutation may cause a change in the folding (3-dimensional structure) of the peptide and/or its proper association of other peptides in a multimeric protein.

The term “trinucleotide repeat” refers to a class of mutations that overlap with the chromosomal disorders, since large deletions in the “trinucleotide repeat” can be seen using cytological methods. A trinucleotide repeat is a 3-base-pair sequence of nucleic acid (typically DNA) in or around the gene which is reiterated tandemly (one directly adjacent to the next) multiple times. The mutation is observed when abnormal expansion of the repeat at variable levels results in the abnormal phenotype. The severity of the disorder can sometimes be correlated with the number of repeats in the expanded region, e.g., fragile X mental retardation syndrome, Huntington Disease, and myotonic dystrophy.

The base of interest, i.e. the base to be identified, may be a “single-nucleotide polymorphism” (SNP), which refers to any nucleotide sequence variation, preferably one that is common in a population of organisms and is inherited in a Medelian fastion. Typically, the SNP is either of two possible bases, and there is no possibility of finding a third or fourth nucleotide identity at an SNP site.

Thus, a defined nucleotide locus within the target nucleic acid that comprises a base to be identified may contain a point mutation, single nucleotide polymorphism, deletion and/or insertion mutation. The target nucleic acid may also be a complement of such a mutated allele.

The term polymorphism” or “genetic variation, as used herein, refers to the occurrence of two or more genetically determined alternative sequences or alleles in a small region (i.e., one to several (e.g., 2, 3, 4, 5, 6, 7, or 8) nucleotides in length) in a population. The allelic form occurring most frequently in a selected population is referred to as the wild type form. Other allelic forms are designated as variant forms. Diploid organisms may be homozygous or heterozygous for allelic forms.

The genetic variation may be associated with or cause diseases or disorders. The term “associated with,” as used herein, refers to the correlation between the occurrence of the genetic variation and the presence of a disease or a disorder. Such diseases or disorders may be human genetic diseases or disorders and include, but are not limited to, bladder carcinoma, colorectal tumors, sickle-cell anemia, thalassemias, al-antitrypsin deficiency, Lesch-Nyhan syndrome, cystic fibrosis/mucoviscidosis, Duchenne/Becker muscular dystrophy, Alzheimer's disease, X-chromosome-dependent mental deficiency, and Huntington's chorea, phenylketonuria, galactosemia, Wilson's disease, hemochromatosis, severe combined immunodeficiency, alpha-1-antitrypsin deficiency, albinism, alkaptonuria, lysosomal storage diseases, Ehlers-Danlos syndrome, hemophilia, glucose-6-phosphate dehydrogenase disorder, agammaglobulimenia, diabetes insipidus, Wiskott-Aldrich syndrome, Fabry's disease, fragile X-syndrome, familial hypercholesterolemia, polycystic kidney disease, hereditar, spherocytosis, Marfan's syndrome, von Willebrand's disease, neurofibromatosis, tuberous sclerosis, hereditary hemorrhagic telangiectasia, familial colonic polyposis, Ehlers-Danlos syndrome, myotonic dystrophy, osteogenesis imperfecta, acute intermittent porphyria, and von Hippel-Lindau disease.

Target nucleic acids may be amplified before being combined with ODNPs as described below. Any known methods for amplifying nucleic acids may be used. Exemplary methods, such as the use of Qbeta Replicase, Strand Displacement Amplification, transcription-mediated amplification. RACE, and one-sided PCR, are described in detail below.

3. Design of Oligonucleotide Primers (ODNPs)

Methods, kits and compositions of the present invention typically involve or include one or more ODNPs which generally contain a partial IRERS and a region of complementarity with a target nucleic acid. For the purpose of simplicity, the target nucleic acid is described as a single-stranded nucleic acid below. However, one of ordinary skill in the art would readily design the ODNP pair(s) of the present invention wherein the target nucleic acid are double-stranded.

The term “oligonucleotide” (ODN) refers to a nucleic acid fragment (typically DNA or RNA) obtained synthetically as by a conventional automated nucleic acid (e.g., DNA) synthesizer. Oligonucleotide is used synonymously with the term polynucleotide. The term “oligonucleotide primer” (ODNP) refers to any polymer having two or more nucleotides used in a hybridization, extension, and/or amplification reaction. The ODNP may be comprised of deoxyribonucleotides, ribonucleotides, or an analog of either. As used herein for hybridization, extension, and amplification reactions, ODNPs are generally between 8 and 200 bases in length. More preferred are ODNPs of between 12 and 50 bases in length and still more preferred are ODNPs of between 18 and 32 bases in length.

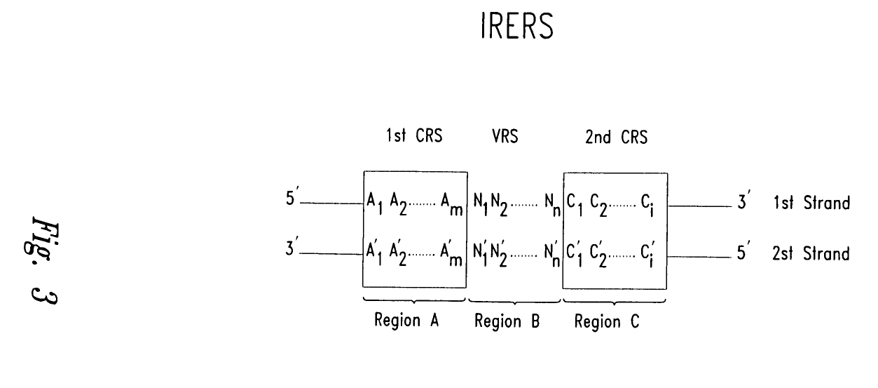

In one aspect, the present invention provides an ODNP useful for producing a portion of a target nucleic acid containing a nucleotide of interest at a defined position. The ODNP comprises an oligonucleotide sequence complementary to a nucleotide sequence of a target nucleic acid at a location 3′ to the defined position. The ODNP further comprises a first CRS of a first strand of an IRERS at a location 3′ to the oligonucleotide sequence complementary to a portion of the target. As described in more detail below, a complete IRERS is a double-stranded oligonucleotide sequence comprising a first CRS and a second CRS linked with a VRS (FIG. 3). The ODNP is so designed that when it anneals to the target, the distance between the nucleotide corresponding to the 3′ terminal nucleotide of the ODNP and the defined position is within the range 0 to n−1 where n is the number of variable nucleotides in the IRERS. Such a design allows the extension product of the ODNP to incorporate a nucleotide complementary to the nucleotide of interest. In a preferred embodiment, the ODNP further comprises one or more nucleotides complementary to the target nucleic acid at the 3′ terminus of the first CRS. The presence of such nucleotides facilitates extension of the primer as the sequence of the first CRS in the ODNP may or may not be exactly complementary to the corresponding nucleotide sequence of the target. In another aspect, the present invention provides an ODNP pair for producing a portion of a target nucleic acid containing a nucleotide to be identified at a defined position. One primer of the ODNP pair (“the first ODNP” or “the forward primer”) comprises a nucleic acid sequence complementary to a nucleotide sequence of a target nucleic acid at a location 3′ to the defined position (“the first region of the target nucleic acid”), whereas the other primer (“the second ODNP” or “the reverse primer”) comprises a nucleic acid sequence complementary to a nucleotide sequence of the complement of the target nucleic acid at a location 3′ to the complementary nucleotide of the nucleotide at the defined position (“the first region of the complement”). The complementarity between the ODNPs and their corresponding target nucleic acid, or the complement thereof, need not be exact, but must be sufficient for the ODNPs to selectively hybridize with the target nucleic acid, or the complement thereof, such that the ODNPs are able to function as primers for extension and/or amplification using the target nucleic acid, or the complement thereof, as a template. Generally, each ODNP contains at least 6, preferably 8, more preferably 10, most preferably 12, 14, or 16 nucleotides that are complementary to the target nucleic acid or the complement thereof. Because each ODNP of the ODNP pair hybridizes to a target nucleic acid, or the complement thereof, at a location 3′ to the defined position in the target or the complementary position in the complement of the target, the resulting extension and/or amplification products from the ODNP pair contains the nucleotide to be identified at the defined position.

Each ODNP in the ODNP pair of the present invention further comprises a partial IRERS, but not a complete IRERS, at a location 3′ to, or preferably at the 3′ terminus of, its nucleic acid sequence described above (i.e. the sequence complementary to the target nucleic acid or the complement thereof). Generally, the first ODNP and the second ODNP comprise the first CRS of the first strand of the IRERS and the second CRS of the second strand of the IRERS, respectively. In addition, the first ODNP and the second ODNP are so spaced that (I) the extension and/or amplification product with the ODNP pair as primers and the target nucleic acid as a template contains a complete IRERS and (2) the nucleic acid to be identified is within the VRS. In other words, the number of nucleotides between the first and the second CRS is the exact number of nucleotides in the VRS so that the extension and/or amplification product from both ODNP can be digested by a RE that recognizes the complete IRERS. The partial IRERS in each ODNP may or may not be complementary to the target nucleic acid.

In a preferred embodiment, each ODNP of the ODNP pair further contains one or more nucleotides that is complementary to the target nucleic acid or the complement thereof (“the second region of the target nucleic acid” and “the second region of the complement,” respectively) at a location 3′ to, or preferably the 3′ terminus of, the CRS. Such nucleotides are a portion of the VRS (FIG. 2). The number of the nucleotides between first and second regions of the target nucleic acid or the complement thereof may be larger or smaller, but preferably equal to, the number of nucleotides of ODNPs between their two regions that are complementary to the target nucleic acids or the complement thereof.

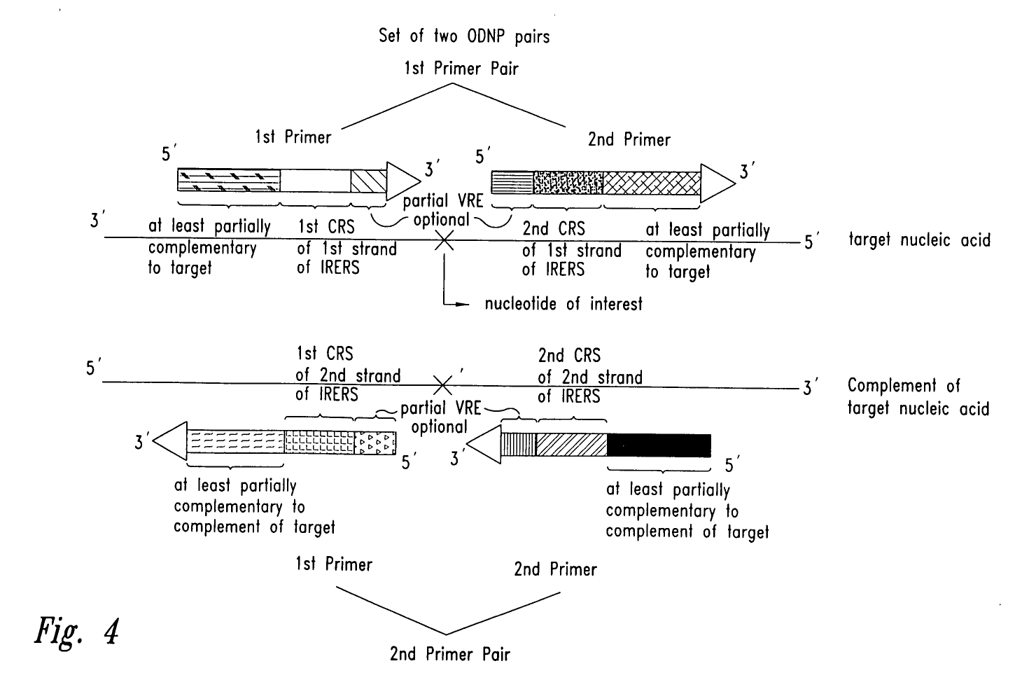

In another aspect, the present invention provides a set of two ODNP pairs for producing a portion of a target nucleic acid containing a nucleotide to be identified at a defined position (FIG. 4). Each pair of the set contain a first ODNP and a second ODNP. The first ODNP of the first ODNP pair comprises an oligonucleotide sequence complementary to a nucleotide sequence of the target nucleic acid at a location 3′ to the defined position. It further comprises a first CRS of a first strand of an IRERS at a location 3′ to, preferably at the 3′ terminus of, the above oligonucleotide sequence. The second ODNP of the first ODNP pair comprises an oligonucleotide sequence complementary to a nucleotide sequence of the target nucleic acid at a location 5′ to the defined position. It further comprises a second CRS of the first stand of the IRERS at a location 5′ to, preferably at the 5′ terminus, of the above oligonucleotide sequence. The first ODNP of the second ODNP pair comprises an oligonucleotide sequence complementary to a nucleotide sequence of the complement of the target nucleic acid at a location 5′ to the position in the complement corresponding to the defined position in the target nucleic acid. It further comprises the first CRS of the second strand of the IRERS at a location 5′ to, preferably at the 5′ terminus of, the above oligonucleotide sequence. The second ODNP of the second ODNP pair comprises an oligonucleotide sequence complementary to a nucleotide sequence of the complement of the target nucleic acid at a location 3′ to the position in the complement corresponding to the defined position in the target. It further comprises the sequence of the second CRS of the second strand of the IRERS at a location 3′ to, preferably at the 3′ terminus of, the above oligonucleotide sequence.

In a preferred embodiment, the first ODNP of the first ODNP pair and the second ODNP of the second ODNP pair each further contains one or more nucleotides that are complementary to a nucleotide sequence of the target nucleic acid or the complement thereof at the 3′ terminus of the first or the second CRS. Such complementarity at the 3′ termini of the ODNPs increases the extension and/or amplification efficiency from the ODNPs.

General techniques for designing sequence-specific primers are well known. For instance, such techniques are described in books, such as PCR Protocols: Current Methods and Application edited by Bruce A. White. 1993: PCR Primer: A Laboratory Manual edited by Carl W. Dieffenbach and Gabriela S. Dveksler. 1995: PCR (Basics: From Background to Bench) by McPherson et al.: PCR Applications: Protocols for Functional Genomics edited by Michael A. Innis. 1999: PCR: Introduction to Biotechniques Series by Neurton and Graham. 1997: PCR Protocols: A Guide to Methods and Applications by Gelfand et al., 1990. PCR Strategies by Michael A. Innis; PCR Technology: Current Innovations, by Griffin and Griffin. 1994; and PCR: Essential Techniques, edited by J. F. Burke. In addition, softwares for designing primers are also available, including Primer Master (see, Proutski and Holmes. Primer Master: A new program for the design and analysis of PCR primmers. Comput. Appl. Biosci. 12: 253-5, 1996) and OLIGO Primer Analysis Software from Molecular Biology Insights. Inc. (Cascade, Colo. USA). The above reference books and description of softwares are incorporated herein by reference in their entireties.

4. Nucleic Acid Hybridization and Extension/Amplification

Methods, kits and compositions of the present invention may involve or include ODNP that are hybridized to the target nucleic acid, where the ODNP facilitates the production and/or amplification of a defined nucleotide locus within the target nucleic acid. The ODNP and target nucleic acid are thus preferably combined under base-pairing condition. Selection of suitable nucleic acid hybridization and/or amplification conditions are available in the art by, e.g, reference to the following laboratory research manuals: Sambrook et al. “Molecular Cloning” (Cold Spring Harbor Press, 1989) and Ausubel et al. “Short Protocols in Molecular Biology” (1999) (incorporated herein by reference in their entirety).

Depending on the application envisioned, the artisan may vary conditions of hybridization to achieve desired degrees of selectivity of ODNP towards target sequence. For applications requiring high selectivity, relatively stringent conditions may be employed to form the hybrids, such as e.g., low salt and/or high temperature conditions, such as from about 0.02 M to about 0.15 M salt at temperatures of from about 50° C. to about 70° C. Such selective conditions are relatively intolerant of large mismatches between the ODNP target nucleic acid.

Alternatively, hybridization of the ODNPs may be achieved under moderately stringent buffer conditions such as, for example, in 10 mM Tris, pH 8.3: 50 mM KCl: 1.5 mM MgCl₂ at 60° C. which conditions permit the hybridization of ODNP comprising nucleotide mismatches with the target nucleic acid. The design of alternative hybridization conditions is well within the expertise of the skilled artisan.

After being hybridized to the target, the ODNPs are extended with the target or the complement thereof as a template using various methodologies known in the art, such as the polymerase chain reaction (PCR) and modified ligase chain reaction (LCR). For the purpose of simplicity, the target nucleic acid is described as a single-stranded nucleic acid below. However, one of ordinary skill in the art would readily extend the ODNPs of the present invention wherein the target nucleic acid are double-stranded (FIGS. 12 and 22).

To obtain a portion of a target nucleic acid containing a defined nucleotide locus and a complete IRERS, at least three runs of extension reaction from the ODNP pair described above need be carried out. Briefly, the first run of extension is for the first primer having a first CRS to incorporate the complement of the nucleotide of interest in the first extension product. The second primer having a second CRS then hybridizes to and extends using the first extension product as a template and thereby incorporate the nucleotide of interest and the first CRS in a second extension product. The first primer then hybridizes to and extends using the second extension product as a template and thereby form, in combination with the second extension product, a double-stranded nucleic acid fragment. Because the first ODNP and the second ODNP of the ODNP pair are spaced in a distance of the same number of base pairs as that of the VRS, the double-stranded nucleic acid fragment resulting from the three runs of extensions contains a complete IRERS.

While three runs of extension reactions are sufficient to produce a fragment containing a defined nucleotide locus within a target nucleic acid and a complete IRERS, preferably, more than three extension reactions are conducted to amplify the fragment. As one of ordinary skill in the art would appreciate, in the subsequent runs of extension, the first primer can hybridize to and extend using any of the target nucleic acid, the second extension product, and the complement of the third extension product as a template, as a template. Similarly, in the subsequent runs of extension, the second primer can hybridize to and extend using either the first extension product or the third extension product as a template. However, because the third extension product and the complement thereof are shorter than any of the target nucleic acid, the first extension product and the second extension product, they are the preferred templates for subsequent extension reactions from either the first or the second ODNPs. This is because the extension efficiency with a short fragment as a template is higher than that with a large fragment as a template. With the increase of the number of extension reactions, the double stranded fragment containing both the nucleotide to be identified and a complete IRERS accumulates quickly than other molecules in the reaction mixture. Such accumulation increases the sensitivity of subsequent characterization of the fragment after being digested with a RE that recognizes the complete IRERS.

The extension/amplification reaction can be carried out known in the art, including PCR methods. For instance. U.S. Pat. Nos. 4,683,195, 4,683,202 and 4,800,159 all describe PCR methods. In addition. PCR methods are also described in several books, e.g., Gelfand et al. PCR Protocols: A Guide to Methods and Application” (1990); Burke (ed), “PCR: Essential Techniques”; McPherson et al. “PCR (Basic: From Background to Bench).” Each of the above references is incorporated herein by reference in its entirety. Briefly, in PCR, two ODNPs are prepared that are complementary to regions on opposite complementary strands of the target nucleic acid sequence. An excess of deoxynucleoside triphosphates is added to a reaction mixture along with a DNA polymerase (e.g. Taq or Pfu polymerase). If the target nucleic acid sequence is present in a sample, the ODNPs will bind to the target and the polymerase will cause the ODNPs to be extended along the target nucleic acid sequence by adding on nucleotides. By raising and lowering the temperature of the reaction mixture, the extended ODNPs will dissociate from the target to form reaction products, excess ODNPs will bind to the target and to the reaction product and the process is repeated.

Exemplary PCR conditions according to the present invention may include, but are not limited to, the following: 100 μl PCR reactions comprise 100 ng target nucleic acid; 0.5 μM of each first ODNP and second ODNP; 10 mM Tris, pH 8.3; 50 mM KCl; 1.5 mM MgCl₂: 200 μM each dNTP; 4 units Taq™ DNA Polymerase (Boehringer Mannheim; Indianapolis, Ind.), and 880 ng TaqStart Tt Antibody (Clontech, Palo Alto, Calif.). Exemplary thermocycling conditions may be as follows: 94° C. for 5 minutes initial denaturation; 45 cycles of 94° C. for 30 seconds, 60° C. for 30 seconds, 72° C. for 1 minute; final extension at 72° C. for 5 minutes. Exemplary nucleic acid polymerases may include one of the thermostable DNA polymerases that are readily available in the art such as. e.g., Taq™, Vent™ or PFU™. Depending on the particular application contemplated, it may be preferred to employ one of the nucleic acid polymerases having a defective 3′ to 5′ exonuclease activity.

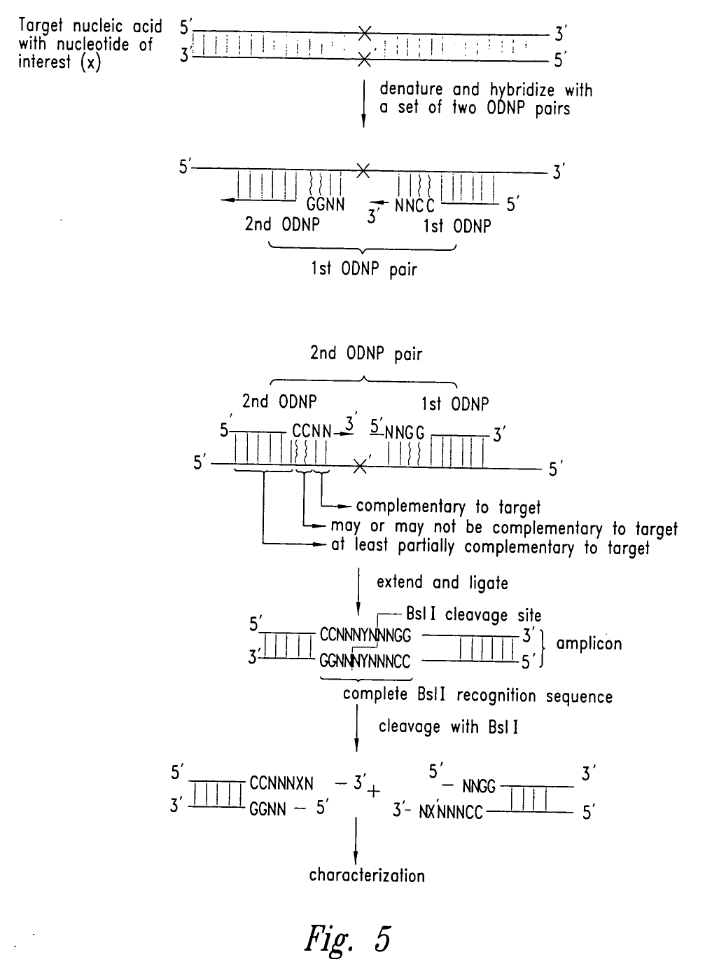

An alternative way to make and/or amplify a fragment containing a nucleotide to be identified and a complete IRERS is by a modified ligase chain reaction, referred to herein as the gap-LCR (Abravava, et al. Nucleic Acids Res. 23:675-682 (1995)), using the set of two ODNP pairs described above (FIG. 5). Briefly, in the presence of the target sequence, each pair of the set will bind to the target, or the complement thereof, located 5′ and 3′ of (on either side of) the nucleotide of interest in the target nucleic acid. In the presence of a polymerase and a ligase, the gap between the two ODNPs of each pair will be filled in and the ODNPs of each pair ligated to form a single unit. By temperature cycling, as in PCR, bound ligated units dissociate from the target and then serve as “target sequences” for ligation of excess ODNP pairs. Thus. LCR uses both a nucleic acid polymerase enzyme and a nucleic acid ligase enzyme to drive the reaction. Exemplary nucleic acid polymerases may include one of the thermostable DNA polymerases that are readily available in the art such as. e.g., Taq™, Vent™ or PFU™. Exemplary nucleic acid ligases may include T4 DNA ligase, or the thermostable Tsc or Pfu DNA ligases. U.S. Pat. No. 4,883,750, incorporated herein by reference in its entirety, describes an alternative method of amplification similar to LCR for binding ODNP pairs to a target sequence.

Exemplary gap-LCR conditions may include, but are not limited to, the following: 50 μl LCR reactions comprise 500 ng DNA; a buffer containing 50 mM EPPS, pH 7.8, 30 mM MgCl₂, 20 mM K⁺. 10 μM NAD, 1-10 μM gap filling nucleotides, 30 nM each oligonucleotide primer. 1 U Thermus flavus DNA polymerase, lacking 3′→5′ exonuclease activity (MBR, Milwukee, Wis.), and 5000 U T. thermophilus DNA ligase (Abbott Laboratories). Cycling conditions may consist of a 30 s incubation at 85° C. and a 30 s incubation at 60° C. for 25 cycles and may be carried out in a standard PCR machine such as a Perkin Elmer 9600 thermocycler.

Another way to provide a double-stranded nucleic acid fragment containing a nucleotide of interest and a complete IRERS is by another modified ligase chain reaction, using two ODNPs and a single-stranded oligonucleotide. The first ODNP comprises an oligonucleotide sequence complementary to a nucleotide sequence of a target nucleic acid at a location 3′ to a nucleotide of interest in the target nucleic acid and a first CRSD of a first strand of an IRERS. The second ODNP comprises an oligonucleotide sequence complementary to a nucleotide sequence of the target at a location 5′ to the nucleotide of interest and a second CRS of the first strand of the IRERS. In the present of the target, a DNA polymerase and a DNA ligase, the two ODNPs extend and ligate with each other and the resulting product incorporates a nucleotide complementary to the nucleotide of interest in the target. Such a product is then annealed to a single-stranded oligonucleotide having a sequence complementary to the amplification and ligation product at least within the region from the 5′ terminus of the first ODNP and 3′ terminus of the second ODNP and a universal nucleotide at the position complementary to the nucleotide of interest.

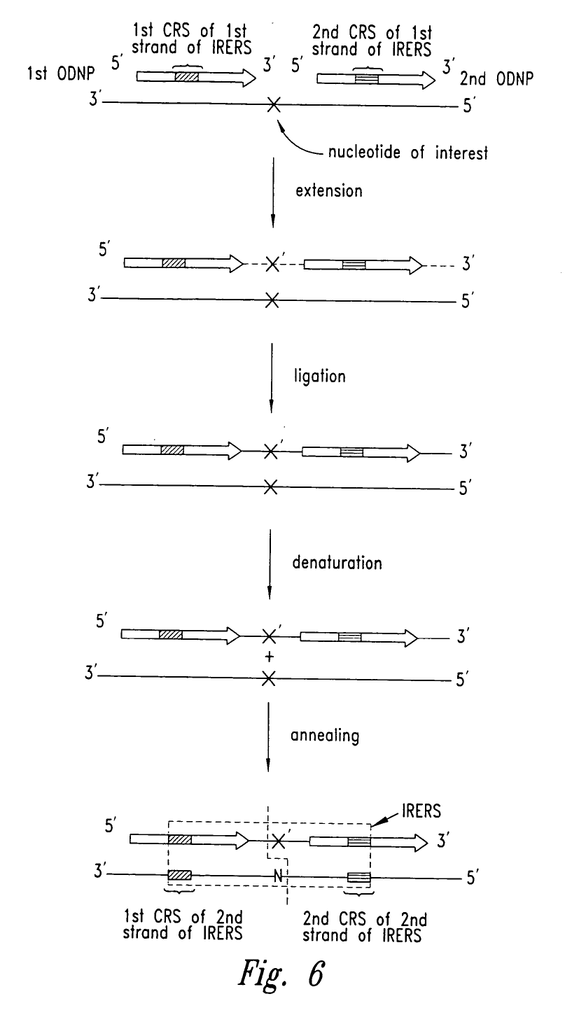

Another way to provide a double-stranded nucleic acid fragment containing a nucleotide to be identified at a defined location in a target nucleic acid and a complete IRERS is illustrated in FIG. 6. A primer pair is mixed with the target. One primer (“the first ODNP”) comprises an oligonucleotide sequence complementary to a nucleotide sequence of the target nucleic acid at a location 3′ to the defined position in the target and a first CRS of a first stand of an IRERS, whereas the other primer (“the second ODNP”) comprises an oligonucleotide sequence complementary to a nucleotide sequence of the target at a location 5′ to the defined position and a second CRS of the first strand of the IRERS. The two primers are then extended using the target as the template to incorporate the complement of the nucleotide to be identified (also referred to as “nucleotide of interest”). The extension products from the two primers are ligated and subsequently disassociated from the target. The disassociated, ligated extension product is then annealed to another nucleic acid molecule that contains the sequence complementary to the ligated extension product in the region from the 5′ terminus of the first ODNP to the 3′ terminus of the second ODNP. This nucleic acid molecule contains a universal nucleotide at a position corresponding to the complement of the nucleotide of interest in the ligated extension product. Such annealing produces a double stranded nucleic acid containing a complete IRERS and the complement of the nucleotide of interest.

In addition to the techniques described above, a number of other template dependent methodologies may be used either to amply target nucleic acids before combining the target nucleic acids with the ODNPs of the present invention. Alternatively, such methodologies may be used, in combination of the ODNP pair or the set of two ODNP pairs described above, to produce a fragment containing a portion of a target nucleic acid with a defined nucleotide locus and a complete IRERS. For instance, Qbeta Replicase, described in PCT Intl. Pat. Appl. Publ. No. PCT/US87/00880, incorporated herein by reference in its entirety, may alternatively be used with methods of the present invention. By this method, a replicative sequence of RNA that has a region complementary to that of a target is added to a sample in the presence of an RNA polymerase. The polymerase will copy the replicative sequence that can then be detected.

Alternatively, Strand Displacement Amplification (SDA) may be employed to achieve isothermal amplification of nucleic acids. By this methodology, multiple rounds of strand displacement and synthesis. i.e. nick translation, are utilized, A similar method, called Repair Chain Reaction (RCR) is another method of amplification which may be useful in the present invention and involves annealing several ODNPs throughout a region targeted for amplification, followed by a repair reaction in which only two of the four bases are present. The other two bases can be added as biotinylated derivatives for easy detection. A similar approach is used in SDA.

Other nucleic acid amplification procedures include transcription-based amplification systems (TAS) (also referred to as transcription-mediated amplification, or TMA) (Kwoh et al. 1989; PCT Intl. Pat. Appl. Publ. No. WO 88/10315, incorporated herein by reference in its entirety), including nucleic acid sequence based amplification (NASBA) and 3SR. In NASBA, the nucleic acids can be prepared for amplification by standard phenol/chloroform extraction, heat denaturation of a sample, treatment with lysis buffer and minispin columns for isolation of DNA and RNA or guanidinium chloride extraction of RNA. These amplification techniques involve annealing an ODNP that has sequences specific to the target sequence. Following polymerization, DNA/RNA hybrids are digested with RNase H while double stranded DNA molecules are heat-denatured again. In either case the single stranded DNA is made fully double stranded by addition of a second target-specific ODNP, followed by polymerization. The double stranded DNA molecules are then multiply transcribed by a polymerase such as one of the RNA polymerases that are readily available in the art, e.g., SP6, T3, or T7. In an isothermal cyclic reaction, the RNAs are reverse transcribed into DNA, and transcribed once again with a polymerase such as T7 or SP6. The resulting products, whether truncated or complete, indicate target-specific sequences.

Eur. Pat. Appl. Publ. No. 329,822, incorporated herein by reference in its entirety, discloses a nucleic acid amplification process involving cyclically synthesizing single-stranded RNA (“ssRNA”), ssDNA, and double-stranded DNA (dsDNA), which may be used in accordance with the present invention. The ssRNA is a first template for a first ODNP, which is elongated by reverse transcriptase (RNA-dependent DNA polymerase). The RNA is then removed from resulting DNA:RNA duplex by the action of ribonuclease H (RNase H, an RNase specific for RNA in a duplex with either DNA or RNA). The resultant ssDNA is a second template for a second ODNP, which also includes the sequences of an RNA polymerase promoter (exemplified by T7 RNA polymerase) 5′ to its homology to its template. This ODNP is then extended by DNA polymerase (exemplified by the large “Klenow” fragment of E. coli DNA polymerase 1), resulting as a double-stranded DNA (“dsDNA”) molecule, having a sequence identical to that of the original RNA between the ODNPs and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

PCT Intl. Pat. Appl. Publ. No. WO 89/06700, incorporated herein by reference in its entirety, disclose a nucleic acid sequence amplification scheme based on the hybridization of a promoter/ODNP sequence to a target single-stranded DNA (“ssDNA”) followed by transcription of many RNA copies of the sequence. This scheme is not cyclic; since new templates are not produced from the resultant RNA transcripts. Other amplification methods include “RACE” (Frohman, 1990), and “one-sided PCR” (Ohara. 1989) which are well-known to those of skill in the art.

5. Restriction Endonucleases and Digestion Conditions

Methods, kits and compositions of the present invention typically involve or include one or more interrupted restriction endonucleases. The term “restriction endonuclease” (RE) refers to the class of nucleases that bind to unique double stranded nucleic acid sequences and that generate a cleavage in the double stranded nucleic acid that results in either blunt, double stranded ends, or single stranded ends with either a 5′ or a 3′ overhang. The “restriction endonuclease recognition sequence (RERS)” is a nucleotide sequence within the double stranded DNA molecule to which the RE binds. The “cleavage site” is the position at which the RE cuts the double stranded DNA molecule.

As used herein, the term “interrupted restriction endonuclease recognition sequence” (IRERS) is defined as a restriction endonuclease recognition site that is comprised of a “first constant recognition sequence (CRS),” a “second CRS,” and a “variable recognition sequence (VRE)” that links the first and second CRSs (FIG. 4). According to the present invention, “first CRS” (also referred to as “Region A”) is defined as that region of the IRERS that contains the constant (not variable) nucleotides of the IRERS that are located 5′ of the VRE of the IRERS. “Second CRS” (also referred to as “Region C”) is defined as that region of the IRERS that contains the constant (not variable) nucleotides of the IRERS that are located 3′ of the VRE of the IRERS. According to the present invention, the “VRE” (also referred as “Region B”) is defined as the stretch of one or more variable nucleotides that are located between the first and second CRSs.

The term “Bsl I” refers to an exemplar RE that binds to a unique nucleic acid sequence that is composed of 5′-CCNNNNNNNGG-3′ where N is an undefined nucleotide base or analog thereof, and that cleaves double-stranded nucleic acid. The cleavage site is as follows:

| | |
|---|---|
| 5′-CCNNNNN/NNGG-3′ | (SEQ ID NO. 1) |
| 3′-GGNN/NNNNNCC-5′ | (SEQ ID NO.2) |

where the bottom and top strands are cleaved 4 bases in from the 3′-OH ends (“/” indicates the cleavage sites). In one aspect of the present invention, the base to be identified. e.g., the mutation or SNP, is positioned within the middle three “Ns” comprising the 3′overhang. In another aspect, the base to be identified is positioned within the 6^{th} nucleotide from the 5′ end of the top strand. Alternatively, the base to be identified may be at any other positions within the variable recognition sequence.

Any restriction endonuclease that recognizes an interrupted restriction endonuclease recognition sequence can be used in the present invention. Some of such enzymes are commercially available from numerous companies such as. e.g., New England Biolabs Inc. (Beverly, Mass.; www.neb.com): Stratagene (La Jolla, Calif.; www.stratagene.com), Promega (Madison, Wis.; www.promega.com), and Clontech (Palo Alto, Calif.; www.clontech.com). Non-commercially available restriction enzymes may be isolated and/or purified based on the teaching available in the art. For instances, the following articles describe the isolation and/or purification of several non-commercially available restriction enzymes suitable for the present invention and are incorporated herein in their entirety by reference: for restriction enzyme ApaB I, Grones and Turna. Biochim. Biophys. Acta 1162:323-325 (1993), Grones and Turna, Biologia (Bratisl) 46:1103-1108 (1991); for EcoH I, Glatman et al., Mol. Gen. Mikrobiol. Virusol. 3:32 (1990); for Fmu I, Rebentish et al. Biotekhnologiya 3:15-16 (1994); for HpyB II, FEMS Microbiol. Lett. 179:175-180 (1999); for Sse8647 I, Nomura et al., European Patent Application No. 0698663 Al. Ishino et al., Nucleic Acids Res. 23: 742-744 (1995), for Unb I, Kawalec et al., Acta Biochim. Pol. 44:849-852 (1997); for VpaK11A 1. Mivahara et al. J. Food Hyg. Sci. Japan 35:605-609 (1994).

Exemplary REs suitable for use in the present invention and their corresponding recognition sequences are presented in Table 1. It will be apparent to one of ordinary skill in the art, however, that REs available in the art that recognizes IRERSs, but are not included in Table 1, may be equally suitable depending on the particular application contemplated.

TABLE 1

Exemplary IRERSs and Their Corresponding REs

| | |
|---|---|
| RE | RECOGNITION SEQUENCE |

| | | |
|---|---|---|
| Ahd I | GACNNN/NNGTC | (SEQ ID NO. 3) |
| AlwN I | CAGNNN/CTG | (SEQ ID NO. 4) |
| Ava II | G/GWCC | (SEQ ID NO. 5) |
| Bgl I | GCCNNNN/NGGC | (SEQ ID NO. 6) |
| Glp I | GC/TNAGC | (SEQ ID NO. 7) |
| Cac8 I | GCN/NGC | (SEQ ID NO. 8) |
| Dde I | C/TNAG | (SEQ ID NO. 9) |
| Dra III | CACNNN/GTG | (SEQ ID NO. 10) |
| EcoN I | CCTNN/NNNAGG | (SEQ ID NO. 11) |
| Hinf I | G/ANTC | (SEQ ID NO. 12) |
| Hpyl66 II | GTNNAC | (SEQ ID NO. 13) |
| Nci I | CC/SGG | (SEQ ID NO. 14) |
| PpuM I | RG/GWCCY | (SEQ ID NO. 15) |
| Sau96 I | G/GNCC | (SEQ ID NO. 16) |
| Sty I | C/CWWGG | (SEQ ID NO. 17) |
| Tfi I | G/AWTC | (SEQ ID NO. 18) |
| Tthlll I | GACN/NNGTC | (SEQ ID NO. 19) |
| Xmn I | GAANN/NNTTC | (SEQ ID NO 20) |

A nucleic acid fragment containing a portion of target nucleic acid with a defined nucleotide locus and a complete IRERS is digested (or cleaved) by a RE that recognizes the IRERS. Conditions for storage and use of restriction endonucleases used according to the present invention are readily available in the art, for example, by reference to one of the laboratory manuals such as Sambrook et al. supra and Ausubel et al. szupra.

Briefly, the number of units of RE added to a reaction may be calculated and adjusted according to the varying cleavage rates of nucleic acid substrates. 1 unit of restriction endonuclease will digest 1 ug of substrate nucleic acid in a 50 μl reaction in 60 minutes. Generally, fragments (e.g., amplicons) may require more than 1 unit/ug to be cleaved completely. The restriction enzyme buffer is typically used at 1× concentration in the reaction. Some restriction endonucleases require bovine serum albumin (BSA) (usually used at a final concentration of 100 μg/ml for optimal activity). Restriction endonucleases that do not require BSA for optimal activity are not adversely affected if BSA is present in the reaction.

Most restriction enzymes are stable when stored at −20° C. in the recommended storage buffer. Exposure to temperatures above −20° C. should be minimized whenever possible. All restriction endonucleases should be kept on ice when not otherwise being stored in the freezer. Enzymes should always be the last component added to a reaction.

The recommended incubation temperature for most restriction endonucleases is about 37° C. Restriction endonucleases isolated from thermophilic bacteria require higher incubation temperatures, typically ranging from 50° C. to 65° C. Incubation time may often be shortened if an excess of restriction endonuclease is added to the reaction. Longer incubation times are often used to allow a reaction to proceed to completion with fewer units of restriction endonuclease.

6. Methodologies for Characterizing Short Nucleic Acid Fragments

The present invention provides methodology whereby a fragment is cleaved using a restriction endonuclease, so as to generate a short (also referred to as “small”) nucleic acid fragment. This short nucleic acid fragment contains information that, upon characterization of the fragment, allows one to determine the identity of the nucleotide(s) of interest in the target nucleic acid. Thus, the present invention transfers information about the nucleotide(s) of interest from a relatively large target nucleic acid into a relatively small nucleic acid fragment. In this way, the nucleotide(s) of interest is made to constitute a relatively large portion of the bases in a nucleic acid, such that characterization of the nucleic acid (fragment) is more readily able to reveal information about the nucleotide(s) of interest. In particular, a direct and complete characterization of the small nucleic acid fragment can be obtained (which is often practically impossible for a large target nucleic acid) which will reveal the identity of the nucleotide(s) of interest.

Thus, as discussed in detail above, methods according to the present invention employ, inter alia, the steps of using appropriate primer(s) and a target nucleic acid to prepare an intermediate structure (e.g., an amplicon) that is digested with a suitable RE (with or without a NE) to produce one or more small nucleic acid fragments. One or more of these fragments that contain either nucleotides of interest or their complement nucleotides are then characterized to obtain partial or complete base sequence information about the fragment to determine the identification of the nucleotides of interest.

The characterization of a nucleic acid fragment (i.e., a digest product) can be done directly, that is, without the need to incorporate a tag or label into the fragment. Alternatively, in some embodiments, it may be advantageous to add one or more detectable labels.

a. Direct Characterization

The present invention transfers information about nucleotide(s) of interest from a relatively large target nucleic acid into a relatively small nucleic acid fragment. Such information transfer allows direct characterization of the small fragment in many instances. For example, small nucleic acid fragments are amenable to direct detection by a variety of mass spectrometric methodologies (as discussed herein below) as well as by ultraviolet (UV) absorption.

In many instances according to the present invention, the complete nucleotide sequence, with the exception of a single nucleotide, will be known for the short nucleic acid fragment even before it is formed. The issue then becomes detecting the nucleotide of interest over the “noise” created while concurrently detecting the other bases. However, if the identity of the other nucleotides is known and their signal in the detection method is known, then this signal can be subtracted from the overall signal for the fragment, to leave information about the nucleotide of interest. This approach is essentially adopted in using mass spectrometry to characterize the small nucleic acid fragment. Other suitable methods, as discussed in detail herein, include determining the mass-to-charge ratio of the small nucleic acid fragment(s), by measuring fluorescence polarization and/or by quantizing ultraviolet (UV) absorption.

In some instances, characterizing a small nucleic acid fragment may entail simply determining the sizes of these single-strand fragments, and from this information the skilled artisan can deduce whether a target nucleic acid contains one or more mutations at a defined nucleotide locus. It will be apparent that the size of a single-strand fragment may be determined by numerous methods that are readily available in the art. Exemplary methods disclosed herein, including methods for measuring the size and/or molecular weight of a single-strand nucleic acid fragment, include, but are not limited to fluorescence including fluorescence polarization (FP), mass spectrometry (MS), ultraviolet (UV) absorption, cleavable mass tags, TaqMan (homogeneous), fluorescence resonance energy transfer (FRET), calorimetric, luminescence and/or fluorescence methodologies employing substrates for horseradish peroxidase (HRP) and/or alkaline phosphatase (AP), as well as methods employing radioactivity.

In certain embodiments of the present invention. Mass Spectrometry (MS) may be employed for characterizing a strand of a small (short) nucleic acid fragments comprising the nucleotide locus of the target nucleic acid. MS may be particularly advantageous in those applications in which it is desirable to eliminate a fractionation step prior to detection. Alternatively, MS may also be employed in conjunction with a fractionation methodology, as discussed herein below, such as, for example, one of the liquid chromatography methodologies including HPLC and DHPLC. Typically, MS detection does not require the addition of a tag or label to the small nucleic acid fragment. Instead, the nucleic acid fragment can be identified directly in the mass spectrometer.

As disclosed herein. MS may be particularly suitable to the detection of small nucleic acid fragments from as small as 1 nucleic acid to as large as several hundred nucleotides. More preferable are fragments of 1 to 50 nucleotides, still more preferable are fragments of from 1 to 14 nucleotides.

Sensitivities may be achieved to at least to 1 amu. The smallest mass difference in nucleic acid bases is between adenine and thymidine, which is 9 Daltons.

Particularly preferred MS methodologies employ Liquid Chromatography-Time-of-Flight Mass Spectrometry (LC-TOF-MS). LC-TOF-MS is composed of an orthogonal acceleration Time-of-Flight (TOF) MS detector for atmospheric pressure ionization (API) analysis using electrospray (ES) or atmospheric pressure chemical ionization (APCI). LC-TOF-MS provides high mass resolution (5000 FWHM), high mass measurement accuracy (to within 5 ppm) and very good sensitivity (ability to detect picomolar amount of DNA polymer) compared to scanning quadrupole instruments. TOF instruments are generally more sensitive than quadrupoles, but correspondingly more expensive.

LC-TOF-MS has a more efficient duty cycle since the current instruments can sequentially analyze one mass at a time while rejecting all others (this is referred to as single ion monitoring (SIM)). LC-TOF-MS samples all of the ions passing into the TOF analyzer at the same time. This results in higher sensitivity and provides quantitative data which improves the sensitivity between 10 and 100 fold. Enhanced resolution (5000 FWHM) and mass measurement accuracy of better than 5 ppm imply that differences between nucleosides as small as 9 amu (Daltons) can be accurately measured. The TOF mass analyzer performs very high frequency sampling (10 spectra/sec) of all ions simultaneously across the full mass range of interest. The duty cycle of the LC-TOF-MS allows high sensitivity spectra to be recorded in quick succession making the instrument compatible with more efficient separation techniques such as narrow bore LC, capillary chromatography (CE) and capillary electrochromatography (CEC). The ions are pulsed into the analyzer, effectively taking a “snapshot” of the ions present at any time.

In the first stage the ES or APCI, aerosol spray is directed perpendicularly past the sampling cone, which is displaced from the central axis of the instrument. Ions are extracted orthogonally from the spray into the sampling cone aperture leaving large droplets, involatile materials, particulates and other unwanted components to collect in the vent port that is protected with an exchangeable liner. The second orthogonal step enables the volume of gas (and ions) sampled from atmosphere to be increased compared with conventional API sources. Gas at atmospheric pressure sampled through an aperture into a partial vacuum forms a freely expanding jet, which represents a region of high performance compared to the surrounding vacuum. When this jet is directed into the second aperture of a conventional API interface it increases the flow of gas through the second aperture. Maintaining a suitable vacuum in the MS-TOF therefore places a restriction on the maximum diameter of the apertures in such an LC interface. Ions in the partial vacuum of the ion block are extracted electrostatically into the hexapole ion bridge which efficiently transports ions to the analyzer.

The coupling of the TOF mass analyzers with MUX-technology allows the connection of up to 8 HPLC columns in parallel to a single LC-TOF-MS. (Micromass, Manchester UK). A multiplexed electrospray (ESI) interface is used for on-line LC-MS utilizing an indexed stepper motor to sequentially sample from up to 8 HPLC columns or liquid inlets operated in parallel.

Use of LC-TOF-MS is generally preferred over use of MALDI-TOF because LC-TOF-MS is a quantitative method for analysis of the molecular weight of polymers. LC-TOF-MS does not fragment the polymers and it employs a very gentle ionization process compared to matrix-assisted-lazer-desorption-ionization (MALDI). Because every MALDI blast is different, the ionization is not quantitative. LC-TOF-MS does, however, produce different m/z values for polymers, but, as disclosed in Example 1 and FIGS. 1-9, this property provides the additional advantage of reducing background and providing complementary information.

Tandem MS or MS/MS is used for structure determination of molecular ions or fragments. In Tandem MS, the ion of interest is selected with the first analyzer (MS-I), collided with inert gas atoms in a collision cell, and the fragments generated by the collision are separated by a second analyzer (MS-2). In Ion Trap and Fourier transform experiments, the analyses are carried out in one analyzer, and the various events are separated in time, not in space. The information can be used to sequence peptides and small DNA/RNA oligomers.

Exact mass measurements, sometimes referred to as “high-resolution measurements,” are used for elemental-composition determination of the sample molecular ion or an ionic fragment. The basis of the method is that each element has a unique mass defect (deviation from the integer mass). The measurement is carried out by scanning with an internal calibrant (in EI or CI mode) or by peak matching (in FAB mode). The elemental composition is determined by comparing the masses of many possible compositions to the measured one. The method is ver reliable for samples having masses up to 800 Da. At higher masses, higher precision or knowledge of expected composition are required to determine the elemental composition unambiguously.

Electron ionization (El) is widely used in mass spectrometry for relatively volatile samples that are insensitive to heat and have relatively low molecular weight. The spectra, usually containing many fragment-ion peaks, are useful for structural characterization and identification. Small impurities in the sample are easy to detect. Chemical ionization (CI) is applied to similar samples: it is used to enhance the abundance of the molecular ion. For both ionization methods, the molecular weight range is 50 to 800 Da. In rare cases it is possible to analyze samples of higher molecular weight. Accuracy of the mass measurement at low resolving power is ±0.1 Dalton and in the high resolution mode, ±5 ppm.

Fast atom bombardment ionization (FAB or sometimes called liquid secondary ionization MS, LSIMS) is a softer ionization method than EI. The spectrum often contains peaks from the matrix, which is necessary for ionization, a few fragments and a peak for a protonated or deprotonated sample molecule. FAB is used to obtain the molecular weight of sensitive, nonvolatile compounds. The method is prone to suppression effects by small impurities. The molecular weight range is 100 to 4000 Da. Exact mass measurement is usually done by peak matching. The accuracy of the mass is the same as obtained in EI, CI.

Matrix-assisted laser desorption (MALDI) has been used to determine the molecular weight of peptides, proteins, oligonucleotides, and other compounds of biological origin as well as of small synthetic polymers. The amount of sample needed is very low (pmoles or less). The analysis can be performed in the linear mode (high mass, low resolution) up to a molecular weight of m/z 300,000 (in rare cases) or reflectron mode (lower mass, higher resolution) up to a molecular weight of 10,000. The analysis is relatively insensitive to contaminants, and accordingly a purification step is not necessarily a part of the characterization process when characterization includes MS. Mass accuracy (0.1 to 0.01%) is not as high as for other mass spectrometry methods. Recent development in Delayed Extraction TOF allows higher resolving power and mass accuracy.

Electrospray ionization (ESI) allows production of molecular ions directly from samples in solution. It can be used for small and large molecular-weight biopolymers (peptides, proteins, carbohydrates, and DNA fragments), and lipids. Unlike MALDI, which is pulsed, it is a continuous ionization method that is suitable for using as an interface with HPLC or capillary electrophoresis. Multiply charged ions are usually produced. ESI should be considered a complement to MALDI. The sample must be soluble, stable in solution, polar, and relatively clean (free of nonvolatile buffers, detergents, salts, etc.).

Electron-capture (sometimes called negative ion chemical ionization or NICI) is used for molecules containing halogens. NO₂ CN, etc. and it usually requires that the analyte be derivatized to contain highly electron-capturing moieties (e.g., fluorine atoms or nitrobenzyl groups). Such moieties are generally inserted into the target analyte after isolation and before mass spectrometric analysis. The sensitivity of NICI analyses is generally two to three orders of magnitude greater than that of PCI or EI analyses. Little fragmentation occurs during NICI.

b. Indirect Characterization

In some embodiments of the present invention, it may be advantageous to add one or more detectable labels to a short nucleic acid fragment or the reaction product thereof (e.g., a portion, or the whole complementary strand of the short nucleic acid fragment). Such labels facilitate the characterization of the fragment and thereby the identification of nucleotide(s) of interest and/or genetic variations within the fragment.

Tables 2 and 3 summarize exemplary labels and detectors, respectively, that are generally suitable for use in methodologies for detecting small nucleic acid fragments.

TABLE 2
Labels Suitable for use in Methodologies for
Detecting Small Nucleic Acid Fragments

| Tagging Technologies | Attributes |
|---|---|
| Fluorophores | Multi-color, overlapping emission spectra, |
| | inexpensive detectors |
| FRET | High sensitivity |
| Fluorescent quenching | Homogenous assay formats |
| Time-resolved fluorescence | Low background |
| Colloidal gold | Good sensitivity |
| Mass Tags (CMSTs) | High level of multiplexing |
| Mass Tags (Electrophore) | High level of multiplexing |
| Radiolabels | Excellent sensitivity |
| Chemiluminescence | Excellent sensitivity |
| Colorimetric | Inexpensive |
| Assay product = “Tag” | Accurate, inexpensive, direct |

TABLE 3
Detectors Suitable for use in Methodologies for
Detecting Small Nucleic Acid Fragments

| Detector | Attributes |
|---|---|
| Film | Inexpensive |
| Scintillation Counter | Reliable, sensitive |
| Fluorescent plate reader | Reliable, inexpensive, sensitive, |
| | multicolor |
| Fluorescence Polarization | Permits homogeneous assay formats. |
| | some instruments very sensitive. |
| Time-resolved fluorescence | Low background. sensitive |
| Fluorescent-monitoring of | Useful information on the process of PCR |
| PCR | |
| ABI-377 | Reliable |
| Capillary Instrument | High throughput, expensive |
| Chemiluminescence plate | Reliable, sensitive |
| reader | |
| CCD | Versatile, sensitive |
| Quadrupole MS | Wide spectral range, quantitative |
| GC/MS | |
| Maldi-TOF | Wide spectral range, not quantitative |
| Plate Reader (colorimetric | Reliable, inexpensive, sensitive |
| assays) | |
| Cell Sorter | High throughput |
| Light Microscopy (Confocal) | Excellent sensitivity |
| Electron mic oscopy | Sensitivity |
| Amphoteric device | Ability to multiplex |
| DHPLC (HPLC/UV) | Reliable, relatively inexpensive |
| HPLC/Fluorescence | Reliable, sensitive, relatively inexpensive |
| Text scanner | Very inexpensive, make your own assay |
| UV box (for stains) | Very inexpensive |

Detectors for these tags and labels are available in generic and non-generic instruments. The generic instruments are the plate readers that usually read micro-plates in 96-well or 384-well formats, and are capable of reading multiple colors (4-6 fluorescent tags). These instruments can be found in customized versions to perform more specialized measurements like time-resolved-fluorescence (TFR) or fluorescence polarization. The detectors for PAGE sequencing and bundled capillary instruments are highly dedicated and non-generic. The generic mass spectrometers MALDI-TOF, electrospray-TOF and APCI-quadrupole (and combinations thereof including ion-trap instruments) are opened-ended instruments with versatility. Suitable software packages have been developed for combinatorial chemistry applications. Scintillation counters are dedicated in that they need to be used with radioisotopes, but can accommodate a wide range of assays formats.

The following is exemplary indirect characterization methodologies. However, the present invention is not limited to these examples. Any techniques known in the art suitable for characterizing small nucleic acid fragments and thereby determining the identity of nucleotide(s) at a defined location may be used in the present invention.

i. Sequencing

In one aspect of the invention, a nucleic acid fragment (i.e. a digestion product described above) is characterized by performing a complete nucleotide sequence analysis. Many techniques are known in the art for identifying each of the bases in a nucleic acid fragment, so as to obtain base sequence information. For instance, two different DNA sequencing methodologies that were developed in 1977, and are commonly known as “Sanger sequencing” and “Maxam Gilbert sequencing,” among other names, are still in wide use today and are well known to those of ordinary skill in the art. See, e.g., Sanger, Proc. Natl. Acad. Sci. (USA) 74:5463, 1977) and Maxam and Gilbert, Proc. Natl. Acad. Sci. (USA) 74:560, 1977). Both methods produce populations of shorter fragments that begin from a particular point and terminate in every base that is found in the nucleic acid fragment that is to be sequenced. The shorter nucleic acid fragments are separated by polyacrylamide gel electrophoresis and the order of the DNA bases (adenine, cytosine, thymine, guanine: also known as A,C,T,G, respectively) is read from a autoradiograph of the gel.

Automated DNA sequencing methods may also be used. Such methods are in wide-spread commercial use to sequence both long and short nucleic acid molecules. In one approach, these methods use fluorescent-labeled primers or ddNTP-terminators instead of radiolabeled components. Robotic components can utilize polymerase chain reaction (PCR) technology which has lead to the development of linear amplification strategies. Current commercial sequencing allows all 4 dideoxy-terminator reactions to be run on a single lane. Each dideoxy-terminator reaction is represented by a unique fluorescent primer (one fluorophore for each base type: A, T, C. G). Only one template DNA (i.e., DNA sample) is represented per lane. Current gels permit the simultaneous electrophoresis of up to 64 samples in 64 different lanes. Different ddNTP-terminated fragments are detected by the irradiation of the gel lane by light followed by detection of emitted light from the fluorophore. Each electrophoresis step is about 4-6 hours long. Each electrophoresis separation resolves about 400-600 nucleotides (nt), therefore, about 6000 nt can be sequenced per hour per sequencer.

Gilbert has described an automated DNA sequencer (EPA. 92108678.2) that consists of an oligomer synthesizer, an array on a membrane, a detector which detects hybridization and a central computer. The synthesizer synthesizes and labels multiple oligomers of arbitrary predicted sequence. The oligomers are used to probe immobilized DNA on membranes. The detector identifies hybridization patterns and then sends those patterns to a central computer which constructs a sequence and then predicts the sequence of the next round of synthesis of oligomers. Through an iterative process, a DNA sequence can be obtained in an automated fashion. This approach may be used to characterize a short nucleic acid fragment (either double or, more commonly single stranded) according to the present invention.

The use of mass spectrometer for the study of monomeric constituents of nucleic acids has also been described (Hignite, In Biochemical Applications of Mass Spectrometry, Waller and Dermer (eds.), Wiley-Interscience. Chapter 16, p. 527, 1972). Briefly, for larger oligomers, significant early success was obtained by plasma desorption for protected synthetic oligonucleotides up to 14 bases long, and for unprotected oligos up to 4 bases in length. As with proteins, the applicability of ESI-MS to oligonucleotides has been demonstrated (Covey et al., Rapid Comm, in Mass Spec. 2:249-256, 1988). These species are ionized in solution, with the charge residing at the acidic bridging phosphodiester and/or terminal phosphate moieties, and yield in the gas phase multiple charged molecular anions, in addition to sodium adducts. These approaches to nucleic acid characterization may be used according to the present invention.

Sequencing nucleic acids with <100 bases by the common enzymatic ddNTP technique is more complicated than it is for larger nucleic acid templates, so that chemical degradation is sometimes employed. However, the chemical decomposition method requires about 50 pmol of radioactive ³²P end-labeled material, 6 chemical steps, electrophoretic separation, and film exposure. For small oligonucleotides (<14 nts), as may need to be characterized according to the present invention, the combination of electrospray ionization (ESI) and Fourier transform (FT) mass spectrometry (MS) is far faster and more sensitive, and is a preferred method for the present invention. Dissociation products of multiply-charged ions measured at high (105) resolving power represent consecutive backbone cleavages providing the full sequence in less than one minute on sub-picomole quantity of sample (Little et al., J. Am. Chem. Soc. 116:4893, 1994). For molecular weight measurements, ESI/MS has been extended to larger fragments (Potier et al., Nuc. Acids Res. 22:3895, 1994). ESI/FTMS appears to be a valuable complement to classical methods for sequencing and pinpoint mutations in nucleotides as large as 100-mers. Spectral data have recently been obtained loading 3×10-13 mol of a 50-mer using a more sensitive ESI source (Valaskoovic. Anal. Chem. 68:259, 1995).

Other methods for obtaining complete, or near complete base sequence information for a nucleic acid molecule are described in the following references: Brennen et al. (Biol. Mass Spec., New York, Elsevier, p. 219, 1990): U.S. Pat. No. 5,403,708): PCT Patent Application No. PCT/US94/02938: and PCT Patent Application No. PCT/US94/11918.

ii. Fluid Handling

As used herein, the term “fluid handling” refers to those assays that are microtiter-plate based and use fluorescence, fluorescence-polarization, luminescence, radioactivity (scintillation counters), or calorimetric readouts. Fluid handling may be useful when the characterization method employs modification of the short nucleic acid fragment. e.g., when a tag or label is incorporated into the short nucleic acid fragment. These assays can be amplified by the use of enzymes such as horseradish peroxidase or alkaline phosphatase that can generate soluble or insoluble calorimetric products from soluble substrates or sensitive luminescent products. These assays have large dynamic ranges (6-8 logs) and can be made robust. Fluid handling using microplates scales well and has been partially miniaturized by the use of 384-well plates. Fluid Handling is especially compatible with commercial robotics and readout systems such as fluorometers, and plate readers. The data is easy to archive and manipulate.

c. Fractionation Methodologies

According to the present invention, the small nucleic acid fragment(s) may, optionally, undergo a step of fractionation prior to a step of detection. The fractionation step may simply remove undesired impurities from the small fragment of interest, to allow more convenient and/or more accurate characterization of the fragment. This type of fractionation step may be referred to as purification. Alternatively, or in addition, the fractionation may separate nucleic acids from one another (such as in chromatography) and the detection technique is simply determining whether the nucleic acid is, or is not, present at a particular time and space (e.g. using ultraviolet detection to determine whether a nucleic acid is eluting from a chromatography column).

Thus, depending on the particular detection methodology employed, it may be advantageous to couple a detection methodology with one or more methodologies for the fractionation of small nucleic acid fragments. As discussed below, such fractionation methodologies include, but are not limited to, electrophoresis including polyacrylamide or agarose gel electrophoresis and capillary electrophoresis, and liquid chromatography (LC) including high pressure liquid chromatography (HPLC) and denaturing high pressure liquid chromatography (DHPLC).

I. Gel Electrophoresis

As used herein, the term “electrophoresis” refers generally to those separation techniques based on the mobility of nucleic acid in an electric field. Negatively charged nucleic acid migrates towards a positive electrode and positively charged nucleic acid migrates toward a negative electrode. Charged species have different migration rates depending on their total charge, size, and shape, and can therefore be separated.

An electrophoresis apparatus consists of a high-voltage power supply, electrodes, buffer, and a support for the buffer such as a polyacrylamide gel, or a capillary tube. Open capillary tubes are used for many types of samples and the other gel supports are usually used for biological samples such as protein mixtures or nucleic acid fragments.

The most powerful separation method for nucleic acid fragments is PAGE, generally in a slab gel format. The major limitation of the current technology is the relatively long time required in performing the gel electrophoresis of nucleic acid fragments produced in sequence reactions. An increased magnitude (10-fold) can be achieved with the use of capillary electrophoresis which utilize ultrathin gels.

Capillary electrophoresis (CE) in its various forms, including free solution, isotachophoresis, isoelectric focusing. PAGE, and micellar electrokinetic “chromatography,” is a suitable technology for the rapid, high resolution separation of very small sample volumes of complex mixtures. In combination with the inherent sensitivity and selectivity of mass spectrometry (CE-MS; see below), CE is a potentially powerful technique for bioanalysis. In the methodology disclosed herein, the interfacing of these two methods provides superior DNA sequencing methods that are superior to the current rate methods of sequencing.

By alternate embodiments, CE may be employed in conjunction with electrospray ionization (ESI) flow rates. The combination of both capillary zone electrophoresis (CZE) and capillary isotachophoresis with quadrapole mass spectrometers based upon ESI have been described. (Olivares et al., Anal. Chem. 59:1230 (1987); Smith et al., Anal. Chem. 60:436 (1988); Loo et al., Anal. Chem. 179:404 (1989); Edmonds et al., J. Chroma. 474:21 (1989); Loo et al., J. Microcolumn Sep. 1:223 (1989); Lee et al., J. Chromatog. 458:313 (1988); Smith et al., J. Chromatog. 480:211 (1989); Grese et al., J. Am. Chem. Soc. 111:2835 (1989) each of which is incorporated herein by reference in its entirety). Small peptides are easily amenable to CZE analysis with good (femtomole) sensitivity.

Polyacrylamide gels, such as those discussed above, may be applied to CE methodologies. Remarkable plate numbers per meter have been achieved with cross-linked polyacrylamide. (See, e.g., Cohen et al. Proc. Natl. Acad. Sci. USA 85:9660 (1988) reporting 10⁺⁷ plates per meter). Such CE columns as described can be employed for nucleic acid (particularly DNA) sequencing. The CE methodology is in principle 25 times faster than slab gel electrophoresis in a standard sequencer. For example, about 300 bases can be read per hour. The separation speed is limited in slab gel electrophoresis by the magnitude of the electric field that can be applied to the gel without excessive heat production. Therefore, the greater speed of CE is achieved through the use of higher field strengths (300 V/cm in CE versus 10 V/cm in slab gel electrophoresis). The capillary format reduces the amperage and thus power and the resultant heat generation.

In alternative embodiments, multiple capillaries may be used in parallel to increase throughput and may be used in conjunction with high throughput sequencing. (Smith et al. Nuc. Acids. Res. 18:4417 (1990); Mathies et al., Nature 359:167 (1992); Huang et al. Anal. Chem. 64:967 (1992); Huang et al. Anal. Chem. 64:2149 (1992)). The major disadvantage of capillary electrophoresis is the limited volume of sample that can be loaded onto the capillary. This limitation may be circumvented by concentrating large sample volumes prior to loading the capillary with the accompanying benefit of >10-fold enhancement in detection.

The most popular method of preconcentration in CE is sample stacking. (Chien et al., Anal. Chem. 64:489A (1992)). Sample stacking depends on the matrix difference (i.e., pH and ionic strength) between the sample buffer and the capillary buffer, so that the electric field across the sample zone is more than in the capillary region. In sample stacking, a large volume of sample in a low concentration buffer is introduced for preconcentration at the head of the capillary column. The capillary is filled with a buffer of the same composition, but at higher concentration. When the sample ions reach the capillary buffer and the lower electric field, they stack into a concentrated zone. Sample stacking has increased detectability by 1-3 orders of magnitude.

Alternatively, preconcentration may be achieved by applying isotachophoresis (ITP) prior to the free zone CE separation of analytes. ITP is an electrophoretic technique that allows microliter volumes of sample to be loaded onto the capillary, in contrast to the low nL injection volumes typically associated with CE. This technique relies on inserting the sample between two buffers (leading and trailing electrolytes) of higher and lower mobility followed by the analyte. The technique is inherently a concentration technique, where the analyses concentrate into pure zones migrating with the same speed. The technique is currently less popular than the stacking methods described above because of the need for several choices of leading and trailing electrolytes, and the ability to separate only cationic or anionic species during a separation process.

Central to the nucleic acid sequencing process is the remarkably selective electrophoretic separation that may be achieved with nucleic acid and/or ODN fragments. Separations are routinely achieved with fragments differing in sequence by only a single nucleotide. This methodology is suitable for separations of fragments up to 1000 bp in length. A further advantage of sequencing with cleavable tags is that there is no requirement to use a slab gel format when nucleic acid fragments are separated by PAGE. Since numerous samples are combined (4 to 2000) there is no need to run samples in parallel as is the case with current dye-primer or dye-terminator methods (i.e., ABI 373 sequencer). Since there is no reason to run parallel lanes, there is no reason to use a slab gel. Therefore, one can employ a tube gel format for the electrophoretic separation method. It has been shown that considerable advantage is gained when a tube gel format is used in place of a slab gel format. (Grossman et al., Genet. Anal. Tech. Appl. 9:9 (1992)). This is due to the greater ability to dissipate Joule heat in a tube format compared to a slab gel which results in faster run times (by 50%), and much higher resolution of high molecular weight nucleic acid fragments (greater than 1000 nt). Long reads are critical in genomic sequencing. Therefore, the use of cleavable tags in sequencing has the additional advantage of allowing the user to employ the most efficient and sensitive nucleic acid separation method that also possesses the highest resolution.

As discussed above, CE is a powerful method for nucleic acid sequencing, particularly DNA sequencing, forensic analysis. PCR product analysis and restriction fragment sizing. CE is faster than traditional slab PAGE since with capillary gels a higher 6+potential field can be applied, but has the drawback of allowing only one sample to be processed per gel. Thus, by alternative embodiments, micro-fabricated devices (MFDs) are employed to combine the faster separations times of CE with the ability to analyze multiple samples in parallel.

MFDs permit an increase in information density in electrophoresis by miniaturizing the lane dimension to about 100 micrometers. The current density of capillary arrays is limited to the outside diameter of the capillary tube. Microfabrication of channels produces a higher density of arrays. Microfabrication also permits physical assemblies not possible with glass fibers and links the channels directly to other devices on a chip. A gas chromatograph and a liquid chromatograph have been fabricated on silicon chips, but these devices have not been widely used. (Terry et al. IEEE Trans. Electron Device ED-26:1880 (1979) and Manz et al. Sens. Actuators B1:249 (1990)). Several groups have reported separating fluorescent dyes and amino acids on MFDs. (Manz et al. J. Chromatography 593:253 (1992): Effenhauser et al., Anal. Chem. 65:2637(1993)).

Photolithography and chemical etching can be used to make large numbers of separation channels on glass substrates. The channels are filled with hydroxyethyl cellulose (HEC) separation matrices. DNA restriction fragments could be separated in as little as two minutes. (Woolley et al., Proc. Natl. Acad Sci. 91:11348 (1994))

ii. Liquid Chromatography (LC)

Liquid chromatography, including HPLC and DHPLC, may be used in conjunction with one of the detection methodologies discussed above such as, for example, fluorescence polarization, mass spectrometry and/or electron ionization. Alternatively LC, HPLC and/or DHPLC may be utilized in conjunction with a UV detection methodology. Regardless of the detection methodology employed, a fractionation step provides the separation of complex mixtures of non-volatile compounds prior to detection.

LC may be used for compounds that have a high molecular weight or are too sensitive to heat to be analyzed by GC. The most common ionization methods that are interfaced to LC are ESI and Atmospheric Chemical Ionization (APCI) in positive and negative-ion modes. The LC is done in most cases by RP-HPLC, and the buffer system should not contain involatile salts (e.g., phosphates). ESI can be used for m/z 500-4000 and is done at low resolving power. LC-MS can be used to look at a wide variety of biologically important compounds including, peptides, proteins, oligonucleotides, and lipids.

The chromatography for gene expression profiling or genotyping by LC/MS can be performed using a ProStar Helix System (catalog X Helixsys01) which is composed of two pumps, a column oven, a UV detector, a degasser, a mixer and an autoinjector. The column is like a Varian Microsorb MV (catalog number R0086203F5), C18 packing with 5 uM particle size, with 300 Angstroms pore size, 4.6 mm×50 mm. The column can be run at 30° C. to 40° C. with a gradient of acetonitrile in 100 mM Triethylamine acetate (TEAA) and 0.1 mM EDTA. The following HPLC method can be used to separation the fragments on the column: Buffer A is 100 mM TEAA with 0.1 mM EDTA, Buffer B is 100 mM TEAA with 0.1 mM EDTA and 25% (V/V) acetonitrile. 0-3 minutes there is a gradient of 20% B to 25% B, at 3.01 minutes to 4 minutes, there is a ramp to 45% B, at 4.01 to 4.5 minutes there is a ramp to 95% B, at 4.51 minutes there is 1 minutes hold at 20% B to re-equilibrate the column. The column can be run at 30-50C by adjusting the column oven to 30C to 50C. The flow rate can be 0.5 to 1.5 ml per minute. About 1 to 200 nanogram of fragment can be injected per 10-50 microliter volume. The UV detector measures the effluent of the column.

High-Performance Liquid Chromatography (HPLC) is a chromatographic technique for separation of compounds dissolved in solution. HPLC instruments consist of a reservoir of mobile phase, a pump, an injector, a separation column, and a detector. Compounds are separated by injecting an aliquot of the sample mixture onto the column. The different components in the mixture pass through the column at different rates due to differences in their partitioning behavior between the mobile liquid phase and the stationary phase. The pumps provide a steady high performance with no pulsating, and can be programmed to vary the composition of the solvent during the course of the separation.

Exemplary detectors useful within the methods of present invention include UV-VIS absorption, or fluorescence after excitation with a suitable wavelength, mass spectrometers and IR spectrometers. Oligonucleotides labeled with fluorochromes may replace radio-labeled oligonucleotides in semi-automated sequence analysis, minisequencing and genotyping. (Smith et al., Nature 321:674 (1986)).

IP-RO-HPLC on non-porous PS/DVB particles with chemically bonded alkyl chains may be employed in the analysis of both single and double-strand nucleic acids. (Huber et al., Anal. Biochem. 212:351 (1993); Huber et al., Nulc. Acids Res. 21:1061 (1993); Huber et al., Biotechniques 16:898 (1993)). In contrast to ion-exchange chromatography, which does not always retain double-strand DNA as a function of strand length (since AT base pairs interact with the positively charged stationary phase, more strongly than GC base-pairs), IP-RP-HPLC enables a strictly size-dependent separation.

A method has been developed using 100 mM triethylammonium acetate as ion-pairing reagent, phosphodiester oligonucleotides could be successfully separated on alkylated non-porous 2.3 μM poly(styrene-divinylbenzene) particles by means of high performance liquid chromatography. (Oefner et al. Anal. Biochem. 223:39 (1994)). The technique described allows the separation of PCR products differing by only 4 to 8 base pairs in length within a size range of 50 to 200 nucleotides.

Denaturing HPLC (DHPLC) is an ion-pair reversed-phase high performance liquid chromatography methodology (IP-RP-HPLC) that uses a non-porous C-18 column as the stationary phase. The column is comprised of a polystyrene-divinylbenzene copolymer. The mobile phase is comprised of an ion-pairing agent of triethylammonium acetate (TEAA), which mediates binding of DNA to the stationary phase, and acetonitrile (ACN) as an organic agent to achieve subsequent separation of the DNA from the column. A linear gradient of acetonitrile allows separation DHPLC identifies mutations and polymorphisms based on detection of heteroduplex formation between mismatched nucleotides in double stranded PCR amplified DNA. Sequence variation creates a mixed population of heteroduplexes and homoduplexes during reannealling of wild type and mutant DNA of fragments based on size and/or presence of heteroduplexes (this is the traditional use of the DHPLC technology). When this mixed population is analyzed by HPLC under partially denaturing temperatures, the heteroduplexes elute from the column earlier than the homoduplexes because of their reduced melting temperature. Analysis can be performed on individual samples to determine heterozygosity, or on mixed samples to identify sequence variation between individuals.

In certain applications, it may be preferred to use the DHPLC column in a non-denaturing mode in order to separate identically sized DNA fragments which possess a different nucleotide composition. For example, the non-denaturing mode may be applicable where, for example, a 6-mer contains a C→T single nucleotide polymorphism (SNP) such as where the wild-type single strand DNA fragment has the nucleotide sequence 5′-AACCCC-3′ and where the mutant single strand DNA fragment has the nucleotide sequence 5′-AATCCC-3′. Fragments as short as 1-mers. 2-, 3-, 4-, 5-, 6-, 7-, 8-, to 6-mers show different mobilities (retention times) on the DHPLC instrument. Alternative to applications employing non-porous materials for performing the chromatography of the small nucleic acid fragments generated by IPRE cleavage, HPLC as both sizing and DHPLC applications work on a wide pore silica based material. Porous materials have the advantage of high sample capacity for semipreparative work. This is marketed by HP as Eclipse dsDNA columns.

7. Software for Analysis of Sequence Information Derived from Detection Methodologies

Detection methodologies employed in the methods of the present invention may optionally employ one or more computer algorithms for analyzing the derived sequence information. Algorithms of the present invention may be encompassed within software packages that convert a detection signal, such as a mass-to-charge ratio of a given small nucleic acid fragment, to a genotyping call.

Exemplary software packages may comprise the following: a peak identification algorithm which identifies peaks above a certain threshold of intensity (area under the curve), an algorithm that identifies and records the mass to charge ratio of the peaks between the scan intervals, an algorithm that calculates the intensity of peaks by measuring the area under the curve, an algorithm that calculates the number of peaks during a scan interval, an algorithm that calculates the ratio of each set of two peaks, an algorithm that calculates the allele calling from the ratiometric values.

The software package and algorithms may record the sample identification (sample ID), source, primer name and sequence, mass to charge ratio of expected fragment, estimation of expected mass to charge ratio, mass spectrometry details, sample plate ID, sample well ID, date and time, number of peaks observed, observed mass to charge ratio, and calculated allele call. The algorithms may also download the data to existed databases and check for accuracy of recording.

A complete genotyping system for use with a mass spectrometry detection system can comprise one or more components listed in the following: A computer (eg., a Dell Optiplex Gx 110, with a CD-ROM), a software package to control the mass spectrometry, a thermocycler, and a robot that moves microtiter plates on and off the autoinjector, a simple HPLC to desalt the PCR or amplification reaction, the mass spectrometer such as an Agilent LC-quadrupole. ES-TOF, a Micromass ES-TOF or APCI-quadrupole, and a software program to call the alleles.

The software package that converts the mass to charge ratio of the fragment to a genotyping call is composed of the following: an algorithm that records the temporal parameter of the chromatography, a peak identification algorithm which identifies peaks above a certain threshold of intensity (area under the curve), an algorithm that identifies and records the mass to charge ratio of the peaks between the scan intervals, an algorithm that calculates the intensity of peaks by measuring the area under the curve, an algorithm that calculates the number of peaks during a scan interval, an algorithm that calculates the ratio of each set of two peaks, an algorithm that calculates the allele calling from the ratiometric values. The software package and algorithms record the sample identification (sample ID), source, primer name and sequence, mass to charge ratio of expected fragment, estimation of expected mass to charge ratio, chromatography details, elution time of each fragments, mass spectrometry details, sample plate ID, sample well ID, date and time, number of peaks observed, observed mass to charge ratio, and calculated allele, sequence identity, or gene identity call. The algorithms will also download the data to existed databases and check for accuracy of recording.

C. Applications for the Methods, Compositions and Compounds of the Present Invention in the Detection of Mutations and Defined Nucleotide Loci

As discussed in detail herein above, the present invention provides methodology for the detection of mutations at defined nucleotide loci within target nucleic acids and/or measurement of genetic variations in parallel. Also provided herein, are various “readout” technologies that may be employed with the methodologies of the present invention for detecting, for example, the size and/or molecular weight of one or more single-strand fragment comprising the mutations and/or genetic variations. Methods according to the present invention will find utility in a wide variety applications wherein it is necessary to identify such a mutation at a defined nucleotide locus or measure genetic variations. Such applications include, but are not limited to, genetic analysis for hereditary diseases, tumor diagnosis, disease predisposition, forensics or paternity, crop cultivation and animal breeding, expression profiling of cell function and/or disease marker genes, and identification and/or characterization of infectious organisms that cause infectious diseases in plants or animals and/or that are related to food safety. Furthermore, the present methods may be utilized to greatly increase the specificity, sensitivity and throughput of the assay while lowering costs in comparison to conventional methods currently available in the art. Described below are certain exemplary applications of the present invention.

1. Expression Profiling

Most mRNAs are transcribed from single copy sequences. Another property of cDNAs is that they represent a longer region of the genome because of the introns present in the chromosomal version of most genes. The representation varies from one gene to another but can be very significant as many genes cover more than 100 kb in genomic DNA, represented in a single cDNA. One possible use of molecular profiling is the use of probes from one species to find clones made from another species. Sequence divergence between the mRNAs of mouse and man permits specific cross-reassociation of long sequences, but except for the most highly conserved regions, prevents cross-hybridization of PCR primers.

Differential screening in complex biological samples such as developing nervous system using cDNA probes prepared from single cells is now possible due to the development of PCR-based and cRNA-based amplification techniques. Several groups reported previously the generation of cDNA libraries from small amounts of poly (A)+RNA (1 ng or less) prepared from 10-50 cells (Belyav et al., Nuc. Acids Res. 17:2919, 1989). Although the libraries were sufficiently representative of mRNA complexity, the average cDNA insert size of these libraries was quite small (<2 kb).

More recently, methodologies have been combined to generate both PCR-based (Lambolez et al. Neuron 9:247, 1992) and cRNA-based (Van Gelder et al. Proc. Natl. Acad Sci. USA 87:1663, 1990) probes from single cells. After electrical recordings, the cytoplasmic contents of a single cell were aspirated with patch-clamp microelectrodes for in situ cDNA synthesis and amplification. PCR was used to amplify cDNA of selective glutamate receptor mRNAs from single Purkinje cells and GFAP mRNA from single glia in organotypic cerebellar culture (Lambolez et al. Neuron 9:247, 1992). In the case of cRNA amplification, transcription promoter sequences were designed into primers for cDNA synthesis and complex antisense cRNAs were generated by in vitro transcription with bacteriophage RNA polymerases.

The methods of the present invention are useful for determining whether a particular cDNA molecule is present in cDNAs from a biological sample and further determine whether genetic variation(s) exist in the cDNA molecule.

2. Forensics

The identification of individuals at the level of DNA sequence variation offers a number of practical advantages over such conventional criteria as fingerprints, blood type, or physical characteristics. In contrast to most phenotypic markers. DNA analysis readily permits the deduction of relatedness between individuals such as is required in paternity testing. Genetic analysis has proven highly useful in bone marrow transplantation, where it is necessary to distinguish between closely related donor and recipient cells. Two types of probes are now in use for DNA fingerprinting by DNA blots. Polymorphic minisatellite DNA probes identify multiple DNA sequences, each present in variable forms in different individuals, thus generating patterns that are complex and highly variable between individuals. VNTR probes identify single sequences in the genome, but these sequences may be present in up to 30 different forms in the human population as distinguished by the size of the identified fragments. The probability that unrelated individuals will have identical hybridization patterns for multiple VNTR or minisatellite probes is very low. Much less tissue than that required for DNA blots, even single hairs, provides sufficient DNA for a PCR-based analysis of genetic markers. Also, partially degraded tissue may be used for analysis since only small DNA fragments are needed. The methods of the present invention are useful in characterizing polymorphism of sample DNAs, therefore useful in forensic DNA analyses. For example, the analysis of 22 separate gene sequences in a sample, each one present in two different forms in the population, could generate 1010 different outcomes, permitting the unique identification of human individuals.

3. Tumor Diagnostics

The detection of viral or cellular oncogenes is another important field of application of nucleic acid diagnostics. Viral oncogenes (v-oncogenes) are transmitted by retroviruses while their cellular counterparts (c-oncogenes) are already present in normal cells. The cellular oncogenes can, however, be activated by specific modifications such as point mutations (as in the c-K-ras oncogene in bladder carcinoma and in colorectal tumors), small deletions and small insertions. Each of the activation processes leads, in conjunction with additional degenerative processes, to an increased and uncontrolled cell growth. In addition, point mutations, small deletions or insertions may also inactivate the so-called “recessive oncogenes” and thereby leads to the formation of a tumor (as in the retinoblastoma (Rb) gene and the osteosarcoma). Accordingly, the present invention is useful in detecting or identifying the point mutations, small deletions and small mutations that activate oncogenes or inactivate recessive oncogenes, which in turn, cause cancers.

4. Transplantation Analyses

The rejection reaction of transplanted tissue is decisively controlled by a specific class of histocompatibility antigens (HLA). They are expressed on the surface of antigen-presenting blood cells, e.g., macrophages. The complex between the HLA and the foreign antigen is recognized by T-helper cells through corresponding T-cell receptors on the cell surface. The interaction between HLA, antigen and T-cell receptor triggers a complex defense reaction which leads to a cascade-like immune response on the body.

The recognition of different foreign antigens is mediated by variable, antigen-specific regions of the T-cell receptor—analogous to the antibody reaction. In a graft rejection, the T-cells expressing a specific T-cell receptor which fits to the foreign antigen, could therefore be eliminated from the T-cell pool. Such analyses are possible by the identification of antigen-specific variable DNA sequences which are amplified by PCR and hence selectively increased. The specific amplification reaction permits the single cell-specific identification of a specific T-cell receptor.

Similar analyses are presently performed for the identification of auto-immune disease like juvenile diabetes, arteriosclerosis, multiple sclerosis, rheumatoid arthritis, or encephalomyelitis.

Accordingly, the present invention is useful for determining gene variations in T-cell receptor genes encoding variable, antigen-specific regions that are involved in the recognition of various foreign antigens.

5. Genome Diagnostics

Four percent of all newborns are born with genetic defects: of the 3,500 hereditary, diseases described which are caused by the modification of only a single gene, the primary molecular defects are only known for about 400 of them.

Hereditary diseases have long since been diagnosed by phenotypic analyses (anamneses, e.g., deficiency of blood: thalassemias), chromosome analyses (karyotype, e.g., mongolism: trisomy 21) or gene product analyses (modified proteins. e.g., phenylketonuria: deficiency of the phenylalanine hydroxylase enzyme resulting in enhanced levels of phenylpyruvic acid). The additional use of nucleic acid detection methods considerably increases the range of genome diagnostics.

In the case of certain genetic diseases, the modification of just one of the two alleles is sufficient for disease (dominantly transmitted monogenic defects); in many cases, both alleles must be modified (recessively transmitted monogenic defects). In a third type of genetic defect, the outbreak of the disease is not only determined by the gene modification but also by factors such as eating habits (in the case of diabetes or arteriosclerosis) or the lifestyle (in the case of cancer). Very frequently, these diseases occur in advanced age. Diseases such as schizophrenia, manic depression or epilepsy should also be mentioned in this context; it is under investigation if the outbreak of the disease in these cases is dependent upon environmental factors as well as on the modification of several genes in different chromosome locations.

Using direct and indirect DNA analysis, the diagnosis of a series of genetic diseases has become possible: bladder carcinoma, colorectal tumors, sickle-cell anemia, thalassemias, al-antitrypsin deficiency, Lesch-Nyhan syndrome, cystic fibrosis/mucoviscidosis, Duchenne/Becker muscular dystrophy, Alzheimer's disease, X-chromosome-dependent mental deficiency, and Huntington's chorea, phenylketonuria, galactosemia, Wilson's disease, hemochromatosis, severe combined immunodeficiency, alpha-1-antitrypsin deficiency, albinism, alkaptonuria, lysosomal storage diseases, Ehlers-Danlos syndrome, hemophilia, glucose-6-phosphate dehydrogenase disorder, agammaglobulimenia, diabetes insipidus, Wiskott-Aldrich syndrome, Fabry's disease, fragile X-syndrome, familial hypercholesterolemia, polycystic kidney disease, hereditary spherocytosis, Marfan's syndrome, von Willebrand's disease, neurofibromatosis, tuberous sclerosis, hereditary hemorrhagic telangiectasia, familial colonic polyposis, Ehlers-Danlos syndrome, myotonic dystrophy, osteogenesis imperfecta, acute intermittent porphyria, and von Hippel-Lindau disease. The present invention is useful in diagnosis of any genetic diseases that are caused by point mutations, small deletions or small insertions at defined positions.

6. Infectious Disease

The application of recombinant DNA methods for diagnosis of infectious diseases has been most extensively explored for viral infections where current methods are cumbersome and results are delayed. In situ hybridization of tissues or cultured cells has made diagnosis of acute and chronic herpes infection possible. Fresh and fomalin-fixed tissues have been reported to be suitable for detection of papillomavirus in invasive cervical carcinoma and in the detection of HIV, while cultured cells have been used for the detection of cytomegalovirus and Epstein-Barr virus. The application of recombinant DNA methods to the diagnosis of microbial diseases has the potential to replace current microbial growth methods if cost-effectiveness, speed, and precision requirements can be met. Clinical situations where recombinant DNA procedures have begun to be applied include the identification of penicillin-resistant Neisseria gonorrhoeae by the presence of a transposon, the fastidiously growing chlamydia, microbes in foods: and simple means of following the spread of an infection through a population. The worldwide epidemiological challenge of diseases involving such parasites as leishnania and plasmodia is already being met by recombinant methods.

The present invention is useful to detect and/or measure genetic variations that are involved in infectious diseases, especially those in drug resistance genes. Thus, the present invention facilitates the characterization and classification of organisms that cause infectious diseases and consequently the treatment of such diseases caused by these organisms.

The following example is provided by way of illustration and not limitation.

### EXAMPLE

### SEPARATION OF GENOTYPING FRAGNIENTS GENERATED BY BSL I DIGESTION AND SUBSEQUENT ANALYSIS BY LIQUID CHRONIATOGRAPHY AND DETECTION WITH A UV DETECTOR AND TIME OF FLIGHT MASS SPECTROMETER

The following example describes the amplification of a specific sequence from the human genome in which the primers contain the Bsl I restriction endonuclease recognition sequence. The resulting amplicon contains a cutting site that liberates a two double-strand oligonucleotide fragments, which is then subjected to a chromatography step and identified by mass to charge ratio.

The 50 μl PCR reactions were composed of 25 ng genomic DNA. 0.5 μM each forward and reverse primers, 10 mM Tris pH 8.3, 50 mM KCl, 1.5 mM MgCl₂, 200 μM each dNTP, 1 Unit DNA Polymerase (MasterAmp™ Taq DNA Polymerase from Epicentre Technologies, Madison Wis, or Vent exo-Polymerase New England BioLabs, Beverly Mass.). Thermocycling conditions were as follows: 95° C. for 3 minutes followed by 30 cycles of 92° C. for 40 seconds, 60° C. for 30 seconds, 72° C. for 30 seconds. A MJ Research PTC-100 thermocycler (MJ Research, Watertown, Mass.) was used for all PCR reactions. Primers were purchased from MWG Biotech (High Point, N.C.).

After the thermocycling was complete, an enzyme mixture was prepared containing Bsl I and 10× Bsl I buffer (New England BioLabs Beverly, Mass.). The mixture was added to each well to make final concentrations of 150 mM KCl, 10 mM Tris-HCl, 2 mM MgCl₂, 1 mM DTT, pH 7.5. The reaction was carried out at 55° C. for more than 60 minutes. The reaction mixture was injected directly without any further manipulation.

The chromatography system is an Varian Prostar Helix system composed of a binary pump, degasser, a column oven, a diode array detector, and thermostatted microwell plate autoinjector (Varian Inc. Walnut Creek, Calif.). The column is a Varian Microsorb MV, incorporating C18 packing with 3 uM particle size, with 300 Angstrom pore size, 2.1 mm×50 mm (Varian Inc. Walnut Creek, Calif.). The column was run at 30C with a gradient of acetonitrile in 5 mM Triethylamine acetate (TEAA). Buffer A is 5 mM TEAA, buffer B is 5 mM TEAA and 25% (V/V) acetonitrile. The gradient begins with a hold at 110% B for one minute, then ramps to 50% B over 4 minutes followed by 30 seconds at 95% B and finally returning to 110% B for a total run time of six minutes. The column temperature was held constant at 30° C. The flow rate was 0.416 ml per minute. The injection volume was 10 microliters. The flow rate into the mass spectrometer was 200ul/min, and half of the LC flow was diverted to waste using a tee. The mass spectrometer is a Micromass LCT Time-of-Flight with an electrospray inlet (Micromass Inc. Manchester UK). The samples were run electrospary negative mode with a scan range from 700 to 2300 amu using an one second scan time. Instrument parameters were: TDC start voltage 700, TDC stop voltage 50. TDC threshold 0. TDC gain control 0. TDC edge control 0, Lteff 1117.5. Veff 4600. Source parameters are desolvation gas 862 L/hr, capillary 3000V, sample cone 25V. RF lens 200V, extraction cone 2V, desolvation temperature 250C. source temperature 150C. RF DC offset 1 4V. FR DC offset 2 1V. Aperture 6V, accelaration 200V, focus 10V, steering 0V, MCP detector 2700V, pusher cycle time (manual) 60, ion energy 40V, tube lens 0V, grid 2 74V, TOF flight tube 4620V, and reflectron 1790V.

The cytochrome 2D6 gene containing a specific Single Nucleotide Polymorphism (T→deletion) was tested and successfully separated and identified. A partial sequence of the gene surrounding the SNP is shown below with the location of the polymorphism italicized and in bold face and the region that functions as a template in an amplification reaction using a pair of internal primers (described below) underlined:

agcagaggcg cttctccgtg tccaccttgc gcaacttggg cctgggcaag aagtcgctgg agcagtgggt gaccgaggag gccgcctgcc tttgtgccgc cttcgccaac cactccggtg ggtgatgggc (SEQ ID NO. 21)

Two primer pairs are used for amplifying the region of cytochrome 2D6 gene containing the SNP. The external primers are designed to amplify only cytochrome 2D6 gene, not its pseudogenes. The internal primers are designed to have a partial Bsl I recognition sequence and to amplify a small region of cytochrome 2D6 gene containing the SNP. The sequences of these two primer pairs as well as that of the final amplification product are shown below with the bases that will form the restriction sites highlighted in bold face. Some or all the bases in the restriction sites may be mismatched with the template sequence.

| | | |
|---|---|---|
| External primer forward: | 5′-GAG ACC AGG GGG AGC ATA-3′ | (SEQ ID NO. 22) |
| External primer reverse: | 5′-GGC GAT CAC GTT GCT CA-3′ | (SEQ ID NO. 23) |
| Internal forward primer: | 5′-TGGGCCTGGATGCTAAGTCGCTGGCCCAG-3′ | (SEQ ID NO. 24) |
| Internal reverse primer: | 5′-GGC CTC CTC GGT CCC CC-3′ | (SEQ ID NO. 25) |
| Final amplification product: | tgggcctggatgctaagtcgctggcccagtgggggaccgaggaggcc | (SEQ ID NO. 26) |

The final amplification product is then digested by Bsl I and denatured. One of the digestion product has the sequence tgggcctggatgctaagtcgctggcccagtg (SEQ ID NO. 27). The mass of this sequence, including a 3′ OH and 5′ PO4 is 9993.2 amu.

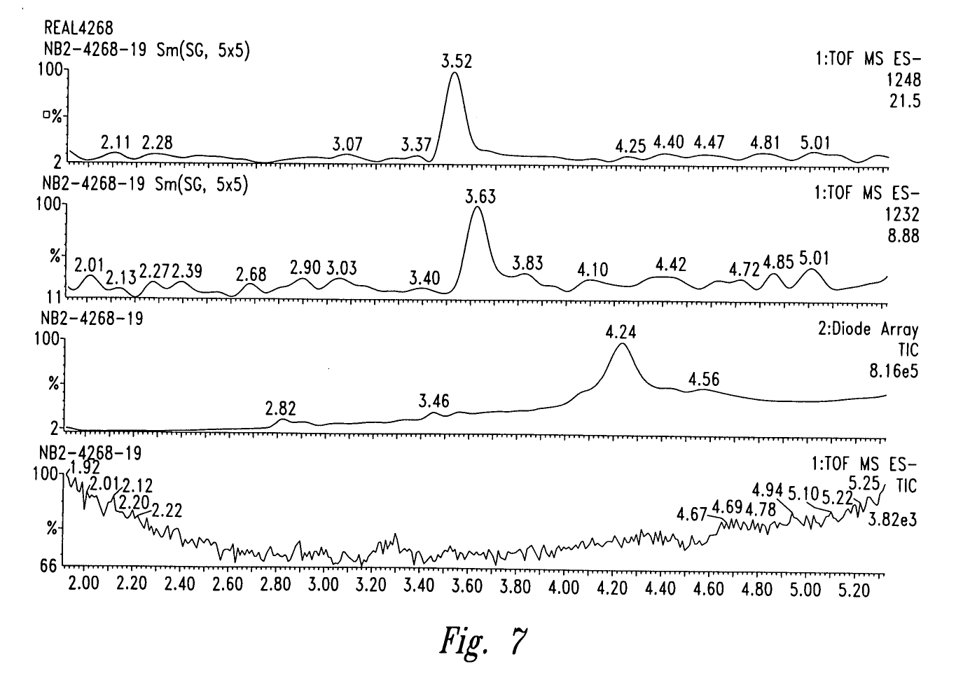

In FIG. 7, the UV chromatogram is shown. The top panel shows the genotyping fragment with an M/Z value of 1246 (8 charges) representing the wild type allele. The TOF was calibrated to a +2 amu the day of the measurement giving the ionized fragment a mass of 1248. Since the MS is run in negative mode, an M/Z value of 1248 is observed for a mass of 1249. The extracted mass of the fragment is 9993 daltons. The second panel is the positive control of 1232 for (8 charges) to calibrate the M/Z measurements. The third panel is the UV trace and the bottom panel shows the total ion current.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A method for identifying a nucleotide at a defined position in a single-stranded target nucleic acid, comprising
(a) forming a mixture of a first oligonucleotide primer (ODNP), a second ODNR, and the target nucleic acid, wherein
the first ODNP comprises a nucleotide sequence that is complementary to a nucleotide sequence of the target nucleic acid at a location 3′ to the defined position,
the second ODNP comprises a nucleotide sequence that is complementary to a nucleotide sequence of the complement of the target nucleic acid at a location 3′ to the complementary nucleotide of the nucleotide at the defined position, and
the first and second ODNPs further comprise a first constant recognition sequence (CRS) of a first strand and a second CRS of a second strand of an interrupted restriction endonuclease recognition sequence (IRERS), respectively but not a complete IRERS, the complete IRERS being a double-stranded nucleic acid having the first and the second strands and comprising the first and the second constant recognition sequences (CRS) linked by a variable recognition sequence (VRS);
(b) extending the first and second ODNPs to form a fragment having the complete IRERS wherein the nucleotide to be identified is within the VRS;
(c) cleaving the fragment with a restriction endonuclease that recognizes the complete IRERS; and
(d) characterizing a product of step (c) to thereby determine the identity of the nucleotide at the defined position.

2. The method of claim 1 wherein the defined position is polymorphic.

3. The method of claim 1 wherein a mutation at the defined position is associated with a disease.

4. The method of claim 3 wherein the disease is a human genetic disease.

5. The method of claim 1 wherein a mutation at the defined position is associated with drug resistance of a pathogenic microorganism.

6. The method of claim 1 wherein the single-stranded target nucleic acid is one strand of a denatured double-stranded nucleic acid.

7. The method of claim 6 wherein the double-stranded nucleic acid is genomic nucleic acid.

8. The method of claim 6 wherein the double-stranded nucleic acid is cDNA.

9. The method of claim 1 wherein the single-stranded target nucleic acid is derived from the genome of a pathogenic virus.

10. The method of claim 1 wherein the single-stranded target nucleic acid is derived from the genome or episome of a pathogenic bacterium.

11. The method of claim 1 wherein the target nucleic acid is synthetic nucleic acid.

12. The method of claim 1 wherein the nucleotide sequence of the first ODNP complementary to the target nucleic acid is at least 12 nucleotides in length.

13. The method of claim 1 wherein the nucleotide sequence of the second ODNP complementary to the complement of the target nucleic acid is at least 12 nucleotides in length.

14. The method of claim 1 wherein the first ODNP is 15-85 nucleotides in length.

15. The method of claim 1 wherein the second ODNP is 15-85 nucleotides in length.

16. The method of claim 1 wherein the first ODNP further comprises one or more nucleotides complementary to the target nucleic acid at the 3′ terminus of the first CRS.

17. The method of claim 1 wherein the second ODNP further comprises one or more nucleotides complementary to the target nucleic acid at the 3′ terminus of the second CRS.

18. The method of claim 1 wherein step (b) comprises performing a polymerase chain reaction.

19. The method of claim 1 wherein step (d) is performed at least partially by the use of a technique selected from the group consisting of mass spectrometry, liquid chromatography, fluorescence polarization, electron ionization, gel electrophoresis, and capillary electrophoresis.

20. The method of claim 1 wherein step (d) is performed at least partially by the use of mass spectrometry.

21. The method of claim 1 wherein step (d) is performed at least partially by the use of liquid chromatography.

22. The method of claim 1 wherein step (d) is performed at least partially by the use of electron ionization.

23. The method of claim 1 wherein step (d) is performed at least partially by the use of gel electrophoresis.

24. The method of claim 1 wherein step (d) is performed at least partially by the use of capillary electrophoresis.

25. The method of claim 1 wherein all of steps (a) through (d) are performed in a single vessel.

26. The method of claim 1 wherein the IRERS is recognizable by a restriction endonuclease selected from the group consisting of Bsl I, Mwo I, and Xcm I.

27. An oligonucleotide primer, comprising
(a) a first CRS of a first strand of an IRERS, but not the first strand of a complete IRERS, the complete IRERS being a double-stranded oligonucleotide having the first strand and a second strand and comprising the first CRS and a second CRS linked by a VRS, the VRS having a number n of variable nucleotides: and
(b) at a location 5′ to the 5′ terminus of the first CRS, an oligonucleotide sequence complementary to a nucleotide sequence of a single-stranded target nucleic acid at a location 3′ to a defined position, wherein when the oligonucleotide sequence anneals to the target nucleic acid, the distance between the nucleotide in the target corresponding to the 3′ terminal nucleotide of the primer and the defined position is within the range 0 to n-1.

28. The primer of claim 27 wherein oligonucleotide sequence (b) is at least 12 nucleotides in length.

29. The primer of claim 27 wherein the primer is 15-85 nucleotides in length.

30. The primer of claim 27 wherein the primer further comprises one or more nucleotides complementary to the target nucleic acid at the 3′ terminus of the first CRS.

31. The oligonucleotide primer of claim 27 wherein the IRERS is recognizable by Bsl I.

32. The primer of claim 27 wherein the defined position in the target nucleic acid is polymorphic.

33. The primer of claim 27 wherein a mutation at the defined position in the target nucleic acid is associated with a disease.

34. The primer of claim 27 wherein the target nucleic acid is one strand of a denatured double-stranded nucleic acid.

35. The primer of claim 34 wherein the double-stranded nucleic acid is genomic nucleic acid.

36. The primer of claim 34 wherein the double-stranded nucleic acid is cDNA.

37. An oligonucleotide primer pair for producing a portion of a single-stranded target nucleic acid containing a nucleotide to be identified at a defined position, comprising first and second ODNPs wherein
the first ODNP comprises a nucleotide sequence complementary to a nucleotide sequence of the target nucleic acid at a location 3′ to the defined position;
the second ODNP comprises a nucleotide sequence complementary to a nucleotide sequence of the complement of the target nucleic acid at a location 3′ to the complementary nucleotide of the nucleotide to be identified;
the first and second ODNPs further comprise a first constant recognition sequence (CRS) of a first strand and a second CRS of a second strand of an interrupted restriction endonuclease recognition sequence (IRERS), respectively, but not a complete IRERS, the complete IRERS being a double-stranded nucleic acid having the first and the second strands and comprising the first and the second constant recognition sequences (CRS) linked by a variable recognition sequence (VRS); and
a fragment resulting from an amplification of the first and second ODNPs comprises a complete IRERS, wherein the nucleotide to be identified is within the VRE.

38. The primer pair of claim 37 wherein the nucleotide sequence complementary to the target nucleic acid of the first ODNP is at least 12 nucleotides in length.

39. The primer pair of claim 37 wherein the nucleotide sequence complementary to the complement of the target nucleic acid of the second ODNP is at least 12 nucleotides in length.

40. The primer pair of claim 37 wherein either the first ODNP or the second ODNP is 15-85 nucleotides in length.

41. The primer pair of claim 37 wherein the first ODNP further comprises one or more nucleotides complementary to the target nucleic acid at the 3′ terminus of the first CRS.

42. The primer pair of claim 37 wherein the second ODNP further comprises one or more nucleotides complementary to the target nucleic acid at the 3′ terminus of the second CRS.

43. The primer pair of claim 37 wherein the IRERS is recognizable by Bsl I.

44. The primer pair of claim 37 wherein the defined position in the target nucleic acid is polymorphic.

45. The primer pair of claim 37 wherein a mutation at the defined position in the target nucleic acid is associated with a disease.

46. The primer pair of claim 37 wherein the target nucleic acid is one strand of a denatured double-stranded nucleic acid.

47. The primer pair of claim 37 wherein the double-stranded nucleic acid is genomic nucleic acid.

48. The primer pair of claim 37 wherein the double-stranded nucleic acid is cDNA.

49. A composition comprising the primer according to any one of claims 27-36 and the target nucleic acid.

50. A kit comprising the primer pair according to any one of claims 37-48.

51. The kit of claim 50 further comprises a restriction endonuclease that recognizes the IRERS.

52. The kit of claim 50 further comprises instruction of use thereof.

53. A set of two ODNP pairs, comprising first and second ODNP pairs each comprising first and second ODNPs wherein:
(a) the first ODNP in the first ODNP pair comprises
an oligonucleotide sequence complementary to a nucleotide sequence of a single-stranded target nucleic acid at a location 3′ to a defined position in the target nucleic acid, and
a first CRS of a first strand of an IRERS, but not the first strand of a complete IRERS, the complete IRERS being a double-stranded nucleic acid having first and second strands and comprising the first CRS and a second CRS linked by a VRS;
(b) the second ODNP in the first ODNP pair comprises
an oligonucleotide sequence complementary to a nucleotide sequence of the target nucleic acid at a location 5′ to the defined position, and
a second CRS of the first strand of the IRERS, but not the first strand of the complete IRERS;
(c) the first ODNP in the second ODNP pair comprises
an oligonucleotide sequence complementary to a nucleotide sequence of the complement of the target nucleic acid at a location 5′ to the position in the complement corresponding to the defined position in the target nucleic acid, and
a first CRS of the second strand of the IRERS, but not the second strand of the complete IRERS; and
(d) the second ODNP in the second ONDP pair comprises
an oligonucleotide sequence complementary to a nucleotide sequence of the complement of the target nucleic acid at a location 3′ to the position in the complement corresponding to the defined position in the target nucleic acid, and
a second CRS of the second strand of the IRERS, but not the second strand of the complete IRERS; and
(e) a fragment resulting from an extension and ligation of the first and second ODNPs in each ODNP pair comprises the complete IRERS, wherein the nucleotide to be identified is within the VRS.

54. A method comprising:
(a) providing a double-stranded nucleic acid molecule comprising an interrupted restriction endonuclease recognition sequence (IRERS), wherein the IRERS comprises a first constant recognition sequence (CRS) and a second CRS linked by a variable recognition sequence (VRS), the VRS having a nucleotide of interest;
(b) cleaving the nucleic acid molecule with a restriction endonuclease that recognizes the IRERS; and
(c) characterizing at least one of the products of step (b) to determine the identity of the nucleotide of interest.

55. The method of claim 54, wherein at least one of the products of step (b) is characterized by a technique selected from liquid chromatograph, mass spectrometry, electron ionization, gel electrophoresis, and capillary electrophoresis.

56. The method of claim 54, wherein the restriction endonuclease is Bsl I.

57. The method of claim 54, wherein step (a) comprises
(i) forming a mixture of the primer pair set of claim 68 and the target nucleic acid;
(ii) extending the first and second ODNPs of the first and second ODNP pairs:
(iii) ligating the extended products of step (b); and
(iv) amplifying the fragments of step (c).

58. The method of claim 54, wherein step (a) comprises
(i) forming a mixture of the primer pair of claim 46 and the target nucleic acid; and
(ii) extending the first and the second ODNPs.

59. The method of claim 54, wherein step (a) comprises
(i) forming a mixture of a first ODNP, a second ODNP and a single-stranded target, wherein
the first ODNP comprises
an oligonucleotide sequence complementary to a nucleotide sequence of the target nucleic acid at a location 3′ to a defined position in the a target nucleic acid, and
a first CRS of a first strand of an IRERS, but not the first strand of a complete IRERS, the complete IRERS being a double-stranded nucleic acid having first and second strands and comprising the first CRS and a second CRS linked by a VRS,
the second ODNP comprises
an oligonucleotide sequence complementary to a nucleotide sequence of the target nucleic acid at a location 5′ to the defined position, and
a second CRS of the first strand of the IRERS, but not the first strand of the complete IRERS;
(ii) extending the first and second ODNPs;
(iii) ligating the extended products of step (ii); and
(iv) annealing the ligation product of step (iii) with an oligonucleotide wherein the oligonucleotide has a universe nucleotide at the position corresponding to the defined position in the target nucleic acid and the resulting double-stranded nucleic acid molecule comprising an IRERS.

60. A method comprising the steps:
(a) combining a first ODNP, a second ODNP, and a target nucleic acid under primer extension conditions, wherein
the first ODNP comprises
an oligonucleotide sequence complementary to a nucleotide sequence of the target nucleic acid at a location 3′ to a defined position in the a target nucleic acid, and
a first CRS of a first strand of an IRERS, but not the first strand of a complete IRERS, the complete IRERS being a double-stranded nucleic acid having first and second strands and comprising the first CRS and a second CRS linked by a VRS,
the second ODNP comprises
an oligonucleotide sequence complementary to a nucleotide sequence of the target nucleic acid at a location 5′ to the defined position, and
a second CRS of the first strand of the IRERS, but not the first strand of the complete IRERS;
(b) performing at least three rounds of primer extension to provide a primer extension product;
(c) cleaving the primer extension product with a restriction endonuclease that recognizes an interrupted restriction endonuclease recognition sequence (IRERS); and
(d) characterizing at least one of the products of step (c) by a technique selected from liquid chromatography, mass spectrometer electon ionization, gel electrophoresis, and capillary electrophoresis.

61. The method of claims 60 wherein step (b) comprises performing a polymerase chain reaction.

62. The method of claim 60 wherein the target nucleic acid is genomic DNA.

63. The method of claim 60 wherein the target nucleic acid is cDNA.

64. The method of claim 60 wherein all of steps (a) through (c) are performed in a single vessel.

65. The method of claim 60 wherein the restriction endonuclease is Bsl I.
